(19) United States
(12) Patent Application Publication
(10) Pub. No.: US 2004/0132131 A1
(43) Pub. Date: Jul. 8, 2004

(54) Methods for comparitive analysis of carbohydrate polymers and carbohydrate polymers identified using same

(76) Inventors: Ofer Markman, Rehovot (IL); Chana Rothmann-Scherz, Petach Tikva (IL); Yehudit Amor, Jerusalem (IL); Peretz Oron, Seattle, WA (US); Einat Or, Tel Aviv (IL)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/416,044

(22) PCT Filed: Nov. 5, 2001
(86) PCT No.: PCT/US01/47064

(51) Int. Cl. C12P 21/06

(52) U.S. Cl. 435/69.1

(57) ABSTRACT

Disclosed is a method for characterizing a carbohydrate polymer by identifying at least two binding agents that bind to the carbohydrate polymer. Binding is preferably determined by contacting the carbohydrate polymer with substrate that contains a plurality of first saccharide-binding agents affixed at predetermined locations on the substrate. The carbohydrate polymer is allowed to contact the substrate under conditions that allow for formation of a first complex between the first saccharide-binding agent and the carbohydrate polymer. A second saccharide-binding agent, which preferably includes a label, is also contacted with the carbohydrate polymer under conditions that allow for formation of a second complex between the second binding agent and the first complex. Identification of the first and second binding agent allows for characterization of the polysaccharide.

### FIELD OF THE INVENTION

The invention relates generally to a method for analyzing molecules containing polysaccharides and more particularly to a method for analyzing polysaccharides based using saccharide-binding agents such as lectins.

### BACKGROUND OF THE INVENTION

Polysaccharides are polymers that include monosaccharide (sugar) units connected to each other via glycosidic bonds. These polymers have a structure that can be described in terms of the linear sequence of the monosaccharide subunits, which is known as the two-dimensional structure of the polysaccharide. Polysaccharides can also be described in terms of the structures formed in space by their component monosaccharide subunits.

A chain of monosaccharides that form a polysaccharide has two dissimilar ends. One end contains an aldehyde group and is known as the reducing end. The other end is known as the non-reducing end. A polysaccharide chain may also be connected to any of the C1, C2, C3, C4, or C6 atom if the sugar unit it is connected to is a hexose. In addition, a given monosaccharide may be linked to more than two different monosaccharides. Moreover, the connection to the C1 atom may be in either the α or β configuration. Thus, both the two-dimensional and three-dimensional structure of the carbohydrate polymer can be highly complex.

The structural determination of polysaccharides is of fundamental importance for the development of glycobiology. Research in glycobiology relates to subjects as diverse as the identification and characterization of antibiotic agents that affect bacterial cell wall synthesis, blood glycans, growth factor and cell surface receptor structures involved in viral disease, and autoimmune diseases such as insulin dependent diabetes, rheumatoid arthritis, and abnormal cell growth, such as that which occurs in cancer.

Polysaccharides have also been used in the development of biomaterials for contact lenses, artificial skin, and prosthetic devices. Furthermore, polysaccharides are used in a number of non-medical fields, such as the paper industry. Additionally, of course, the food and drug industry uses large amounts of various polysaccharides and oligosaccharides.

In all of the above fields, there is a need for improved saccharide analysis technologies. Saccharide analysis information is useful in, e.g., for quality control, structure determination in research, and for conducting structure-function analyses.

The structural complexity of polysaccharides has hindered their analysis. For example, saccharides are believed to be synthesized in a template-independent mechanism. In the absence of structural information, the researcher must therefore assume that the building units are selected from any of the saccharide units known today. In addition, these units may have been modified, during synthesis, e.g., by the addition of sulfate groups.

Second, saccharide can be connected at any of the carbon moieties, e.g., a the C1, C2, C3, C4, or C6 atom if the sugar unit it is connected to is a hexose. Moreover, the connection to the C1 atom may be in either α or β configuration.

Third, saccharides may be branched, which further complicates their structure and the number of possible structures that have an identical number and kind of sugar units.

A fourth difficulty is presented by the fact that the difference in structure between many sugars is minute, as a sugar unit may differ from another merely by the position of the hydroxyl groups (epimers).

The use of a plurality of such saccharide-binding agents, whether fixed to the substrate and/or employed as the second (soluble) saccharide-binding agent, characterizes the carbohydrate polymer of interest by providing a “fingerprint” of the saccharide. Such a fingerprint can then be analyzed in order to obtain more information about the carbohydrate polymer. Unfortunately, the process of characterization and interpretation of the data for carbohydrate polymer fingerprints is far more complex than for other biological polymers, such as DNA for example. Unlike binding DNA probes to a sample of DNA for the purpose of characterization, the carbohydrate polymer fingerprint is not necessarily a direct indication of the components of the carbohydrate polymer itself. DNA probe binding provides relatively direct information about the sequence of the DNA sample itself, since under the proper conditions, recognition and binding of a probe to DNA is a fairly straightforward process. Thus, a DNA “fingerprint” which is obtained from probe binding can yield direct information about the actual sequence of DNA in the sample.

By contrast, binding of agents to carbohydrate polymers is not nearly so straightforward. As previously described, even the two-dimensional structure (sequence) of carbohydrate polymers is more complex than that of DNA, since carbohydrate polymers can be branched. These branches clearly affect the three-dimensional structure of the polymer, and hence the structure of the recognition site for the binding agent. In addition, recognition of binding epitopes on carbohydrate polymers by the binding agents may be affected by the “neighborhood” of the portion of the molecule which is surrounding the epitope. Thus, the analysis of such “fingerprint” data for the binding of agents to the carbohydrate polymer of interest is clearly more difficult than for DNA probe binding, for example.

A useful solution to this problem would enable the fingerprint data to be analyzed in order to characterize the carbohydrate polymer. Such an analysis would need to transform the raw data, obtained from the previously described process of incubating saccharide-binding agents with the carbohydrate polymer, into a fingerprint, which would itself contain information. The fingerprint would also need to be standardized for comparison across different sets of experimental conditions and for different types of saccharide-binding agents; Unfortunately, such a solution is not currently available.

In spite of these difficulties, a number of methods for the structural analysis of saccharides have been developed. For example, PCT Application No. WO 93/24503 discloses a method wherein monosaccharide units are sequentially removed from the reducing end of an oligosaccharide by converting the monosaccharide at the reducing end to its keto- or aldehyde form, and then cleaving the glycosidic bond between the monosaccharide and the next monosaccharide in the oligosaccharide chain by using hydrazine. The free monosaccharides are separated from the oligosaccharide chain and identified by chromatographic methods. The process is then repeated until all monosaccharides have been cleaved.

PCT Application No. WO 93/22678 discloses a method of sequencing an unknown oligosaccharide by making assumptions upon the basic structure thereof, and then choosing from a number of sequencing tools (such as glycosidases) one which is predicted to give the highest amount of structural information. This method requires some basic information as to the oligosaccharide structure (usually the monosaccharide composition). The method also illustrates the fact that reactions with sequencing reagents are expensive and time-consuming, and therefore there is a need for a method that reduces these expenses.

PCT Application No. WO 93/22678 discloses a method for detecting molecules by probing a monolithic array of probes, such as oligodeoxynucleotides, immobilized on a VLSI chip. This publication teaches that a large number of probes can be bound to an immobilized surface, and the reaction thereof with an analyte detected by a variety of methods, using logic circuitry on the VLSI chip.

European Patent Application No. EP 421,972 discloses a method for sequencing oligosaccharides by labeling one end thereof, dividing the labeled oligosaccharide into aliquots, and treating each aliquot with a different reagent mix (e.g.of glycosidases), pooling the different reaction mixes, and then analyzing the reaction products, using chromatographic methods. This method is useful for N-linked glycans only, as they have a common structure at the point where the saccharide chain is linked to the protein. O-linked glycans are more varied, and the method has as yet not been adapted for such oligosaccharides with greater variability in their basic structure.

There is therefore a need for a system and method for characterizing polysaccharides using an accurate, high throughput method for identifying agents that bind to the polysaccharide.

### SUMMARY OF THE INVENTION

The invention is based in part on the discovery of a method for quickly and accurately identifying agents that bind a given carbohydrate polymer. Also provided by the invention is' a method for generating a fingerprint of a carbohydrate polymer that is based on its pattern of binding to saccharide-binding agents.

In one aspect, the invention features a method for determining the relatedness of a first carbohydrate polymer and a second carbohydrate polymer, e.g., a first glycoprotein and a second glycoprotein or a first polysaccharide and a second polysaccharide. The method includes providing a first fingerprint of a first carbohydrate polymer, wherein the first fingerprint comprises binding information for at least a first saccharide-binding agent and information for a second saccharide-binding agent for the first carbohydrate polymer. A second fingerprint of a second carbohydrate polymer is also provided. The second fingerprint includes binding information for at least the first saccharide-binding agent and the second saccharide-binding agent for the second carbohydrate polymer.

The first fingerprint and the second fingerprint are compared by determining whether the first glycoprotein and the second glycoprotein bind to the first saccharide binding agent, and whether the first glycoprotein and the second glycoprotein bind to the second saccharide binding agent. The similarity between the first and second fingerprint indicate the relatedness of the first glycoprotein and second glycoprotein.

In a further aspect the invention features a method of identifying a carbohydrate polymer, e.g., a glycoprotein, polysaccharide, or glycolipid, by providing a first fingerprint of a test carbohydrate polymer, wherein the first fingerprint comprises binding information for at least a first saccharide-binding agent and information for a second saccharide-binding agent for the first carbohydrate polymer. The first fingerprint is compared to at least one reference fingerprint, wherein the reference carbohydrate polymer fingerprint includes binding information for at least the first saccharide-binding agent and the second saccharide-binding agent for at least one reference carbohydrate polymer. A similar, e.g., identical fingerprint between the first fingerprint and the reference fingerprint indicates that the test carbohydrate polymer is similar, e.g., identical to the reference carbohydrate polymer.

In a still further aspect, the invention includes a method of modifying a carbohydrate polymer, e.g., a glycoprotein, polysaccharide, or glycolipid, by providing a first fingerprint of a test carbohydrate polymer. The first fingerprint comprises binding information for at least a first saccharide-binding agent and binding information for at least a second saccharide-binding agent for the first carbohydrate polymer.

The first fingerprint is compared to at least one reference fingerprint. The reference fingerprint can include binding information for at least the first saccharide-binding agent and information for the second saccharide-binding agent for the reference carbohydrate polymer. Differences between the first fingerprint and the reference fingerprint are identified. The test carbohydrate polymer is then modified so that its fingerprint is increased or decreased, as desired, with respect to the fingerprint of the reference carbohydrate polymer.

Also included in the invention is a method of synthesizing a carbohydrate polymer-containing compound, e.g., a glycoprotein. For example, in one embodiment the invention includes making a glycoprotein by providing a polypeptide and or attaching carbohydrate polymers to the polypeptide to produce the desired modified glycoprotein.

In a further aspect, the invention features a method for characterizing a carbohydrate polymer. The carbohydrate polymer is contacted with a surface that includes at least one first saccharide-binding agent attached to a predetermined location on the surface under conditions allowing for the formation of a first complex between the first saccharide-binding agent and the carbohydrate polymer. The surface is then contacted with at least one second saccharide-binding agent under conditions allowing for formation of a second complex between the first complex and the second saccharide-binding agent. The first saccharide-binding agent and second saccharide-binding agent are then identified, thereby characterizing the carbohydrate polymer.

Also provided by the invention is a method of generating a fingerprint of a carbohydrate polymer by contacting a carbohydrate polymer with a first saccharide-binding agent, determining whether the carbohydrate polymer binds to the saccharide-binding reagent, contacting the carbohydrate polymer with a second saccharide-binding agent, and determining whether the carbohydrate polymer binds to the second saccharide-binding reagent. Identification of the first and second saccharide-binding agent is used to generate a fingerprint of the carbohydrate polymer.

In preferred embodiments, the fingerprints used in the methods described herein are identified by method that includes providing the carbohydrate polymer and contacting the carbohydrate polymer with the first saccharide-binding agent. A determination is then made as to whether the carbohydrate polymer binds to the first saccharide-binding agent.

The carbohydrate polymer is also contacted with the second saccharide-binding agent, which preferably includes a detectable label. A determination is also made as to whether the carbohydrate polymer binds to the second saccharide-binding agent. The information gathered about the binding of the first saccharide-binding agent and second binding agent is compiled to generate a fingerprint of the carbohydrate polymer.

In more preferred embodiments, binding of the first and second saccharide-agent is determined by providing a surface comprising at least one first saccharide-binding agent attached to a predetermined location on the surface, and contacting the surface with a carbohydrate polymer under conditions allowing for the formation of a first complex between the first saccharide-binding agent and the carbohydrate polymer. The surface is also contacted with at least one second saccharide-binding agent under conditions allowing for formation of a second complex between the first complex and the second saccharide-binding agent. Identification of the second binding agent at a particular location on the surface also allows for the identification of the corresponding first saccharide binding agent attached at that location of the surface.

In another aspect, the invention provides a method of identifying an agent that modulates the structure of a carbohydrate polymer by contacting a biological sample including the with a test agent, and identifying a carbohydrate polymer fingerprint of one or more carbohydrate polymers in the sample. The carbohydrate polymer fingerprint is compared to a carbohydrate polymer fingerprint of the carbohydrate polymers in a sample that is not contacted with the agent. Differences in the carbohydrate fingerprint profiles, if present, are identified in the test and reference fingerprints. A difference in fingerprint profiles indicates the test agent modulates the structure of a carbohydrate polymer.

Also featured by the invention is a method of identifying a candidate therapeutic agent for a pathophysiology associated with a carbohydrate polymer. The method includes providing a test biological sample that includes the carbohydrate polymer and contacting the test biological sample with a test agent. A carbohydrate polymer fingerprint of one or more carbohydrate polymers in the biological sample is identified and compared to a carbohydrate polymer fingerprint of the of one or more carbohydrate polymers in a reference biological sample whose pathophysiological status is known. Differences in the carbohydrate finger profiles, if present, in the test biological sample and reference biological sample, thereby identifying a therapeutic agent for a pathophysiology associated with the carbohydrate polymer.

In a further aspect, the invention features a method of identifying an individualized therapeutic agent suitable for treating a pathophysiology associated with a carbohydrate polymer in a subject by providing from the subject a biological sample that includes the carbohydrate polymer and contacting the test biological sample with a test agent. A carbohydrate polymer fingerprint of one or more carbohydrate polymers in the biological sample is identified and compared to a carbohydrate polymer fingerprint of the one or more carbohydrate polymers in a reference biological sample whose pathophysiological status is known; and identifying a difference in the carbohydrate finger profiles, if present, in the test biological sample and reference biological sample.

Also within the invention is a method of assessing the efficacy of a treatment of pathophysiology associated with a carbohydrate polymer. The method includes providing from the subject a test biological sample including the carbohydrate polymer and determining a carbohydrate fingerprint of the carbohydrate polymer. The carbohydrate fingerprint is compared to a reference carbohydrate polymer fingerprint, wherein the reference carbohydrate polymer fingerprint is derived from a carbohydrate polymer whose pathophysiological status is known, thereby assessing the efficacy of treatment of the pathophysiology in the subject.

Also within the invention is a method of treating a pathophysiology associated with a carbohydrate polymer mediated pathway in a subject, the method comprising administering to the subject an agent that modulates a carbohydrate polymer in the patient, wherein the modulation alters a carbohydrate polymer fingerprint in the patient. The patient is preferably a human patient

Also within the invention is method of identifying an agent that modulates the structure of a carbohydrate polymer. The method includes providing a biological sample that includes the carbohydrate polymer and contacting the sample with a test agent. A carbohydrate polymer fingerprint of one or more carbohydrate polymers in the sample is identified and compared to a carbohydrate polymer fingerprint of the one or more carbohydrate polymers in a sample that is not contacted with the agent. A difference in carbohydrate fingerprint profiles is identified, if present, in the test and reference fingerprints, thereby identifying an agent that modulates the structure of a carbohydrate polymer.

The invention also provides a method of identifying a candidate therapeutic agent for a pathophysiology associated with a carbohydrate polymer by providing a test biological sample comprising a cell capable of expressing the carbohydrate polymer; contacting the test biological sample with a test agent; identifying a carbohydrate polymer fingerprint of one or more carbohydrate polymers in the biological sample; comparing the carbohydrate polymer fingerprint to a carbohydrate polymer fingerprint of one or more carbohydrate polymers in a reference biological sample comprising at least one cell whose pathophysiological status is known; and identifying a difference in the carbohydrate finger profiles, if present, in the test biological sample and reference biological sample, thereby identifying a therapeutic agent for a pathophysiology associated with the carbohydrate polymer.

Also provided herein is a method of identifying an individualized therapeutic agent suitable for treating a pathophysiology associated with a carbohydrate polymer in a subject. The method includes providing from the subject a biological sample comprising the carbohydrate polymer; contacting the test biological sample with a test agent; identifying a carbohydrate polymer fingerprint of one or more carbohydrate polymers in the biological sample; comparing the carbohydrate polymer fingerprint to a carbohydrate polymer fingerprint of the one or more carbohydrate polymers in a reference biological sample whose pathophysiological status is known; and identifying a difference in the carbohydrate finger profiles, if present, in the test biological sample and reference biological sample, thereby identifying an individualized therapeutic agent for the subject.

In a further aspect the invention includes a method of assessing the efficacy of a treatment of pathophysiology associated with a carbohydrate polymer. The method includes providing from the subject a test biological sample comprising the carbohydrate polymer; determining a carbohydrate fingerprint of the carbohydrate polymer; and comparing the carbohydrate fingerprint of the polymer with a reference carbohydrate polymer fingerprint, wherein the reference carbohydrate polymer fingerprint is derived from a carbohydrate polymer whose pathophysiological status is known, thereby assessing the efficacy of treatment of the pathophysiology in the subject.

In a further aspect, the invention includes method of treating a pathophysiology associated with a carbohydrate polymer mediated pathway in a subject by dministering to the subject an agent that modulates activity or levels of a carbohydrate polymer in the patient, wherein the modulation alters a carbohydrate polymer fingerprint in the patient

In preferred embodiments, at least one of the fingerprints identified or utilized in the herein described methods features a plurality of addresses, each address containing a numeric value related to binding of a saccharide-binding agent to the carbohydrate polymer, and the fingerprint is analyzed by a method comprising the steps of: (a) connecting a first address to at least one other address of the fingerprint to form a map; (b) if the first address is consistent with the at least one other address, determining the map to be internally consistent; (c) repeating steps (a) and (b) at least once to form at least one additional map; (d) comparing the map to the at least one additional map to determine if the maps are mutually consistent; and (e) eliminating any mutually inconsistent maps. In preferred embodiments, the method additionally includes the steps of (f) receiving experimental data from a second assay; (g) converting the experimental data to form a second fingerprint; (h) performing steps (a) and (b) with the second fingerprint to form a second fingerprint map; (i) comparing the map to the second fingerprint map to determine if the maps are mutually consistent; and (j) eliminating any mutually inconsistent maps.

If desired, step (g) further may further include (i) analyzing a format of the experimental data; (ii) if the format is not a numerical value format, converting the experimental data to at least one numerical value; and (iii) creating the second fingerprint from the at least one numerical value.

In some embodiments, experimental data for the second assay is obtained by contacting the saccharide-binding agent to a known carbohydrate polymer having at least one of a known function, a known sequence or a combination thereof.

In some embodiments, the second assay is performed under identical experimental conditions as for the carbohydrate polymer.

In some embodiments, the second assay is performed on specific carbohydrate polymer material for the carbohydrate polymer, the specific carbohydrate polymer material being identical as for binding the saccharide-binding agent to the carbohydrate polymer.

In some embodiments, comparing includes integrating external data to the sample carbohydrate polymer fingerprint, the fingerprint featuring a plurality of addresses, each address containing a numeric value related to binding of a saccharide-binding agent to the sample carbohydrate polymer, the method comprising the steps of: (a) converting the external data to form an external fingerprint, the external data including at least one assay being performed on a carbohydrate polymer; (b) comparing the external fingerprint to the fingerprint for the sample carbohydrate polymer; and (c) determining if the external fingerprint is consistent with the fingerprint for the sample carbohydrate polymer.

In some embodiments, step (a) further comprises the steps of: (i) analyzing a format of the external data; (ii) if the format is not a numerical value format, converting the external data to at least one numerical value; and (iii) creating the external fingerprint from the at least one numerical value.

Alternatively, if the format is a numerical value format, the external fingerprint may be created directly from the external data.

In some embodiments, the method further comprises constructing a map for characterizing the carbohydrate polymer by: (a) characterizing the carbohydrate polymer with a fingerprint, the fingerprint featuring a plurality of addresses, each address containing a value obtained from assay data from an experimental assay performed on the carbohydrate polymer;

(b) constructing a plurality of maps according to the fingerprint; (c) obtaining additional data for characterizing the carbohydrate polymer; (d) determining if each map is consistent with the additional data; and (e) if the map is not consistent with the additional data, rejecting the map. Preferably, each map includes a plurality of elements, each element including at least one feature of the carbohydrate polymer being selected from the group consisting of a function of at least a portion of the carbohydrate polymer, a sequence of at least a portion of the carbohydrate polymer, a structure of at least a portion of the carbohydrate polymer, and a combination thereof.

In some embodiments, the carbohydrate polymer features a sequence having a plurality of monosaccharides and step (b) is performed according to sequence information for at least a portion of the sequence, such that the map features at least the portion of the sequence.

In some embodiments, step (b) is performed according to at least one functional epitope of the carbohydrate polymer, the at least one functional epitope being at least a portion of the carbohydrate polymer having a function, such that the map features the functional epitope.

In some embodiments, the carbohydrate polymer features a sequence having a plurality of monosaccharides and step (b) is also performed according to sequence information for at least a portion of the sequence, such that the map features both the functional epitope and at least the portion of the sequence.

Preferably, step (c) is performed with assay data from at least one additional experimental assay performed on the carbohydrate polymer.

In some embodiments, at least one assay is for determining binding of a saccharide-binding agent to the carbohydrate polymer, such that the assay data is obtained from detection of whether binding of the saccharide-binding agent to the carbohydrate polymer occurred.

In some embodiments, the experimental assay is performed on specific carbohydrate polymer material for the carbohydrate polymer, and at least one additional different assay is also performed on the specific carbohydrate polymer material for step (c) for direct comparison of the additional data to the fingerprint.

In preferred embodiments, the carbohydrate polymer features a sequence having a plurality of monosaccharides and wherein step (c) is performed on a known carbohydrate polymer having at least one of a known function, a known sequence or a combination thereof.

In preferred embodiments, the experimental assay is performed on specific carbohydrate polymer material for the carbohydrate polymer, and the experimental assay is also performed on the known carbohydrate polymer for step (c) for direct comparison of the additional data to the fingerprint. In some embodiments, the map is related to an overall characteristic of the carbohydrate polymer.

Preferably, the identifying step further comprises constructing a map for the carbohydrate polymer, the method comprising the steps of: (a) characterizing the carbohydrate polymer according to assay data obtained from at least one experimental assay performed on the carbohydrate polymer; (b) decomposing the assay data into a plurality of addresses, each address featuring a value of the assay data; (c) forming a plurality of maps by connecting each address to at least one other address; and (d) transforming each map into a property vector by correlating the value at each address to a feature of the carbohydrate polymer being selected from the group consisting of a function of at least a portion of the carbohydrate polymer, a sequence of at least a portion of the carbohydrate polymer, a structure of at least a portion of the carbohydrate polymer, and a combination thereof.

In preferred embodiments, step (c) is performed exhaustively to determine all combinations of addresses for maps. Alternatively, or in addition, step (c) is performed recursively.

In preferred embodiments, step (c) is performed by comparing the assay data to at least one template for the property vector, to determine if the feature exists.

The method may further include constructing a map for a carbohydrate polymer by a method that includes the steps of: (a) providing characterizing data for the carbohydrate polymer; (b) deriving a plurality of maps from the characterizing data; (c) obtaining additional data for characterizing the carbohydrate polymer; (d) determining if the additional data is consistent with each of the plurality of maps; (e) if the additional data is not consistent with a map, eliminating the map; and (f) adding an additional map only if the additional map is consistent with the additional data and with each remaining map.

The method may further include characterizing a sample carbohydrate polymer according to a known carbohydrate polymer having at least one of a known function, a known sequence or a combination thereof. The method includes the steps of: (a) performing at least one experimental assay for the sample carbohydrate polymer to obtain assay data; (b) performing an identical experimental assay for the known carbohydrate polymer to obtain comparison assay data; and (c) characterizing the sample carbohydrate polymer according to the known carbohydrate polymer by comparing the assay data to the comparison assay data.

Preferably, at least one experimental assay is performed under identical assay conditions as the identical experimental assay.

In certain preferred embodiments, at least one experimental assay includes at least one assay for determining binding of a saccharide-binding agent to the carbohydrate polymer and to the known carbohydrate polymer.

In preferred embodiments, the carbohydrate polymer fingerprint is identified by a method comprising: providing a first carbohydrate polymer; contacting the first carbohydrate polymer with a first saccharide-binding agent; determining whether the first carbohydrate polymer binds to the first saccharide-binding agent; contacting the carbohydrate polymer with a second saccharide-binding agent, wherein the second saccharide-binding agent comprises a detectable label; and determining whether the first carbohydrate polymer binds to the second saccharide-binding reagent, thereby generating a fingerprint of the carbohydrate polymer.

As disclosed herein, the method may further include contacting the carbohydrate polymer with at least five saccharide-binding agents, and determining whether the carbohydrate polymer binds to each of the at least five saccharide-binding reagents.

In some embodiments, the fingerprints are identified and compared using a system and method for characterizing carbohydrate polymers according to maps obtained from experimental data. Preferably, the data is obtained from a plurality of different types of experimental assays for characterizing the carbohydrate polymer. More preferably, at least one such assay involves binding a saccharide-binding agent to the carbohydrate polymer. One or more features of the carbohydrate polymer is then preferably characterized.

These features are preferably derived from maps of the data obtained from assays involving the sample carbohydrate polymer. These maps are more preferably analyzed at a plurality of levels, with each level providing more abstract biological information. Most preferably, new types of experimental data are introduced to the process of analysis at each level, in order to support more complex analyses of the data. Optionally and most preferably, maps are eliminated at each level as being inconsistent with the experimental data. New maps are most preferably added at a higher level only if they are derived from the new experimental data which has been introduced at that level, in order to prevent a combinatorial explosion at successive levels of data analysis.

According to the present invention, there is provided a method for analyzing a fingerprint for a carbohydrate polymer, the fingerprint featuring a plurality of addresses, each address containing a numeric value related to binding of a saccharide-binding agent to the carbohydrate polymer, the method comprising the steps of: (a) connecting a first address to at least one other address of the fingerprint to form a map; (b) if a value for the first address does not contradict a value for the at least one other address, determining the map to be internally coherent; (c) repeating steps (a) and (b) at least once to form at least one additional map; (d) comparing the map to the at least one additional map to determine if the maps are mutually coherent; and (e) eliminating any mutually inconsistent maps.

Preferably, the method further comprises the steps of: (f) receiving experimental data from a second assay; (g) converting the experimental data to form a second fingerprint; (h) performing steps (a) and (b) with the second fingerprint to form a second fingerprint map; (i) comparing the map to the second fingerprint map to determine if the maps are mutually coherent; and (j) eliminating any mutually inconsistent maps.

More preferably, step (g) further comprises the steps of: (i) analyzing a format of the experimental data; (ii) if the format is not a numerical value format, converting the experimental data to at least one numerical value; and (iii) creating the second fingerprint from the at least one numerical value.

According to another embodiment of the present invention, there is provided a method for integrating external data to a fingerprint for a sample carbohydrate polymer, the fingerprint featuring a plurality of addresses, each address containing a numeric value related to binding of a saccharide-binding agent to the sample carbohydrate polymer, the method comprising the steps of: (a) converting the external data to form an external fingerprint, the external data including at least one assay being performed on a carbohydrate polymer; (b) comparing the external fingerprint to the fingerprint for the sample carbohydrate polymer; and (c) determining if the external fingerprint is consistent with the fingerprint for the sample carbohydrate polymer;

(d) incorporating the external data with the data in the fingerprint to a newly determined fingerprint or “structure vector”.

Hereinafter, the term “glycomolecule” includes any molecule with a polysaccharide component. Examples include polysaccharide, a glycoprotein, and glycolipid.

Hereinafter, the term “saccharide-binding agent” refers to any entity which is capable of binding to a saccharide, whether monosaccharide, oligosaccharide, polysaccharide or a combination thereof, including but not limited to, a lectin, an antibody, another protein which binds to or otherwise recognizes a saccharide, and a polysaccharide-cleaving or modifying enzyme.

Hereinafter, the term “carbohydrate polymer” refers to any polysaccharide or oligosaccharide, or other structure containing a plurality of connected monosaccharide units.

Hereinafter, the term “sample carbohydrate polymer” refers to the carbohydrate polymer under test, for which experimental data is derived for the purposes of further analysis.

Hereinafter, the term “comparison carbohydrate polymer” refers to the carbohydrate polymer for which data is obtained for comparison to the sample carbohydrate polymer. The comparison carbohydrate polymer may optionally be a standard known carbohydrate polymer, for which the structure is known.

Hereinafter, the term “computational device” includes, but is not limited to, personal computers (PC) having an operating system such as DOS, Windows™, OS/2™ or Linux; Macintosh™ computers; computers having JAVA™-OS as the operating system; graphical workstations such as the computers of Sun Microsystems™ and Silicon Graphics™, and other computers having some version of the UNIX operating system such as AIX™ or SOLARIS™ of Sun Microsystems™; or any other known and available operating system, or any device, including but not limited to: laptops, hand-held computers, enhanced cellular telephones such as WAP-enabled cellular telephones, wearable computers of any sort, which can be connected to a network as previously defined and which has an operating system. Hereinafter, the term “Windows™” includes but is not limited to Windows95™, Windows NT™, Windows98™, Windows CE™, Windows2000™, and any upgraded versions of these operating systems by Microsoft Corp. (USA).

For the present invention, a software application could be written in substantially any suitable programming language, which could easily be selected by one of ordinary skill in the art. The programming language chosen should be compatible with the computational device according to which the software application is executed. Examples of suitable programming languages include, but are not limited to, C, C++ and Java.

In addition, the present invention could be implemented as software, firmware or hardware, or as a combination thereof. For any of these implementations, the functional steps performed by the method could be described as a plurality of instructions performed by a data processor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is an illustration of the glycomolecule identity (GMID) cards obtained for pasteurized goat's milk (A and B), non-pasteurized goat's milk (C and D) and bovine milk (E).

FIG. 2 is a reproduction of the GMID cards obtained for various lipopolysaccharide samples. Cards A to E correspond to LPS# 1, 7, 10, 15 and 16 respectively.

FIG. 3 is a high-level logic flowchart that illustrates an algorithm for choosing a set of colored lectins.

FIG. 4 shows an exemplary experimental system for obtaining the raw data for determining a fingerprint for a carbohydrate polymer of interest for the present invention.

FIG. 5 is a flowchart of an exemplary method according to the present invention for comparing the fingerprint of the sample carbohydrate polymer to at least one other fingerprint.

FIG. 6 is a flowchart of an exemplary method according to the present invention for internally analyzing the fingerprint of the sample carbohydrate polymer in order to extend the fingerprint data.

FIG. 7 is a flowchart of an exemplary method according to the present invention for extending the fingerprint data by integration of data from external databases.

FIG. 8 is a flowchart of an exemplary method according to the present invention for locating features of interest within the sample carbohydrate polymer

### DETAILED DESCRIPTION OF THE INVENTION

Provided by the invention are methods for identifying and modifying carbohydrate polymers using information that describes the binding status of the carbohydrate polymers with respect to saccharide-binding agents. The carbohydrate polymer used in the herein describe methods can be any molecule that includes a polysaccharide moiety. Thus, the carbohydrate can be a polysaccharide as well as a molecule to which a polysaccharide is linked (e.g., by a covalent bond) to a second molecule. The second molecule can be, e.g., a sulfate, or a polymer. The carbohydrate polymer can be, e.g., a glycoprotein or a glycolipid. Examples of glycoproteins include growth factors such as erythropoietin (EPO), interferons (including interferon alpha, interferon beta, and interferon gamma), human chronic gonadotropin (hCG), GCSF, antithrombin III, an interleukin, (e.g., IL-2) and hCG.

Examples of polysaccharides include, e.g., glycogen, starch cellulose, heparin, heparin sulfate, fragments of heparin sulfate, and cell wall components such as bacterial lipopolysaccharides or glucans found in yeast cell walls.

General Method for Analysis of Carbohydrate Polymers

In preferred embodiments, the carbohydrate polymers identified, modified, or used in the herein describe methods may be variant forms of a polysaccharide such as, e.g., heparin, or heparin sulfate. For example, variant forms of carbohydrate polymers can be chosen based on desired structural or functional properties of the carbohydrate polymer. For example, variant forms of heparin or heparin sulfate, or fragments of these molecules (such as those produced following cleavage by heparanase) may be selected based on their enhanced ability of the variant form to modulate detachment of the extracellular matrix, to promote cell migration, to bind polypeptides such as chemokines or growth factors, to modulate inflammation, angiogenesis, tumor metastasis, restenosis, or cell proliferation, or to modulate the activity of heparanase.

In one aspect, the invention provides a method for determining the relatedness of a first carbohydrate polymer and a second carbohydrate polymer, e.g. two or more glycoproteins. To determine the relatedness of two or more glycoproteins, a first fingerprint of a first glycoprotein is compared to a second fingerprint of a second glycoprotein. The first fingerprint comprises binding information for at least a first saccharide-binding agent and a second saccharide-binding agent for the first glycoprotein. The second fingerprint comprises binding information for at least the first saccharide-binding agent and the second saccharide-binding agent for the second glycoprotein.

The first fingerprint and the second fingerprint are compared by determining whether the first glycoprotein and the second glycoprotein bind to the first saccharide binding agent, and whether the first glycoprotein and the second glycoprotein bind to the second saccharide binding agent. The degree to which the first and second glycoprotein share the same binding status i.e., binding or non-binding, with respect to the first and second saccharide-binding agents indicates the relatedness of the first glycoprotein and second glycoprotein.

To determine the relatedness of polysaccharides, a first fingerprint of a first polysaccharide is provided. The first fingerprint includes binding information for at least a first saccharide-binding agent and a second saccharide-binding agent for the first polysaccharide. The first fingerprint is compared to a second fingerprint of a second polysaccharide, wherein the second fingerprint comprises binding information for at least the first saccharide-binding agent and the second saccharide-binding agent for the second polysaccharide. The comparing includes determining whether the first polysaccharide and the second polysaccharide bind to the first saccharide binding agent, and whether the first polysaccharide and the second polysaccharide bind to the second saccharide binding agent

Also provided by the invention is a method for identifying a carbohydrate polymer using carbohydrate polymer fingerprint information. For example, in one embodiment, a first fingerprint of a test glycoprotein is provided. The first fingerprint includes binding information for at least a first saccharide-binding agent and a second saccharide-binding agent for the first glycoprotein. The first fingerprint is compared to at least one reference fingerprint, wherein the reference glycoprotein fingerprint comprises binding information for at least the first saccharide-binding agent and the second saccharide-binding agent for at least one reference glycoprotein.

A similarity in fingerprint patterns between the test fingerprint and the reference fingerprint indicates the test glycoprotein and reference glycoprotein are related. For example, identical patterns indicate the test glycoprotein is identical to the reference glycoprotein.

Fingerprint analysis information can also be used to modify glycoproteins to contain, or lack, a desired property. To make a modified glycoprotein, a first fingerprint that includes binding information for at least a first saccharide-binding agent and a second saccharide-binding agent for the first glycoprotein is compared to at least one reference fingerprint. The reference fingerprint includes binding information for at least the first saccharide-binding agent and the second saccharide-binding agent for at least one reference glycoprotein. The status of the reference glycoprotein with respect to the property of interest is preferably known. Differences in the first fingerprint and the reference fingerprint are detected, and this information is used to alter the carbohydrate polymer content of the test glycoprotein to decrease or increase the differences in the first fingerprint and reference fingerprint.

In some embodiments, a fingerprint of the altered test glycoprotein is generated and compared to the reference fingerprint.

Also provided by the invention is a method of synthesizing a glycoprotein by providing a polypeptide that includes the desired amino acid sequence of the glycoprotein and attaching carbohydrate polymers to the polypeptide to produce the desired modified glycoprotein. The polypeptide can be synthesized chemically if desired. Alternatively, the peptide can be recombinantly expressed.

Also within the invention is a carbohydrate polymer produced made by one of the methods described herein. The carbohydrate polymer can be purified using information about its saccharide agent-binding properties. For example, a carbohydrate known to bind to three distinct saccharide agents can be purified using affinity columns that include these agent

The invention also includes a method of diagnosing a pathology associated with a carbohydrate polymer in a subject. To diagnose the pathology, a test fingerprint of a carbohydrate polymer from a subject suspected of having the pathology is compared to a reference fingerprint. The test fingerprint is from a carbohydrate polymer in a reference sample whose pathological state is known. A correspondence between the test fingerprint and the reference fingerprint indicates the subject and the reference sample have the same pathological state. For example, if the reference sample is from a subject (or population of subjects) that does not have the pathology, then a similarity in the fingerprint between the test subject and the reference fingerprint indicates the test subject does not have the pathological state. The reference sample can be drawn from a database.

Also within the invention is a method of identifying a function associated with a carbohydrate polymer by providing a test fingerprint of a carbohydrate polymer from a test sample and comparing the test fingerprint with a reference fingerprint. The test fingerprint is from a carbohydrate polymer whose functional status is known. A correspondence between the test fingerprint and the reference fingerprint indicates the subject and the reference sample have the same functional status.

Identifying Carbohydrate Polymer Fingerprints

A fingerprint can also be used to identify a carbohydrate polymer by comparing a test fingerprint from an unknown carbohydrate polymer sample with a reference fingerprint, which is from carbohydrate polymer whose identity is known. A correspondence between the test fingerprint and the reference fingerprint indicates the subject and the reference carbohydrate sample are the same.

As used herein, a fingerprint of a carbohydrate polymer is a compilation of information about the binding status of the carbohydrate polymer and a plurality of scattered-binding agents. In some embodiments, the fingerprint is a numeric representation of the detection of the presence of binding by the saccharide-binding agents to the carbohydrate polymer.

The fingerprint of the carbohydrate polymer can be generated by contacting the carbohydrate polymer with a first saccharide-binding agent and determining whether the carbohydrate polymer binds to the saccharide-binding reagent. The carbohydrate polymer is also contacted with a second saccharide-binding agent, and a determination is made as to whether the second binding-agent binds to the carbohydrate polymer.

The carbohydrate polymer is preferably contacted with at least five saccharide-binding agents, and a determination is made as to whether the carbohydrate polymer binds to each of the at least five saccharide-binding reagents. In preferred embodiments, the binding of the carbohydrate polymer to at least 10, 15, 20, or 25 or more agents is determined.

In preferred embodiments, binding of the first and second saccharide-agent is determined by providing a surface comprising at least one first saccharide-binding agent attached to a predetermined location on the surface and contacting the surface with a carbohydrate polymer under conditions allowing for the formation of a first complex between the first saccharide-binding agent and the carbohydrate polymer. Unbound polymer is removed if desired, and the surface is contacted with at least one second saccharide-binding agent under conditions allowing for formation of a second complex between the first complex and the second saccharide-binding agent. The first and second saccharide-binding agent are then identified, and the information generated provides a fingerprint for the carbohydrate polymer. By including a plurality of first and/or second saccharide-binding agents, it is possible to generate a detailed fingerprint of the carbohydrate polymer. Of course, it will be apparent to one of ordinary skill in the art that the absence of binding of a first or second saccharide-agent to a carbohydrate polymer will also contribute to the fingerprint generated for the polysaccharide.

The second saccharide agent preferably contains a detectable label. When the second saccharide-binding agent is labeled, the identity of the second label determines the identity of the second saccharide-binding agent. The position of the second label on the substrate in turn reveals the identity of the first saccharide-binding agent.

To assess binding status, the carbohydrate polymer is added to a surface that includes at least one saccharide-binding agent attached to a predetermined location on the surface. The carbohydrate polymer is incubated with the surface under conditions allowing for the formation of a complex between the first saccharide-binding agent and the carbohydrate polymer. The surface can then be washed if desired to remove unbound carbohydrate polymer. The surface is then contacted with a second saccharide-binding agent under conditions allowing for formation of a second complex between the first complex and the second saccharide-binding agent. The second agent preferably carries a detectable label to allow for detection of the second complex. Detection of the second complex at a location on the substrate corresponding to the location of a predetermined binding-agent allows for the identification of the first and second binding agents as agents that bind to the carbohydrate polymer. Detecting the first and second-binding agents provides structural information about the carbohydrate polymer.

While the method has been described by first contacting the carbohydrate polymer with the surface and then adding a detectable label, it is understood that this order is not obligatory. Thus, in some embodiments, the second agent is mixed with the carbohydrate polymer, and this complex is added to the surface.

In some embodiments, a plurality of saccharide-binding agents are attached to the surface. Similarly, a plurality of second detectable saccharide-binding agents may be used. In preferred embodiments, a plurality of both first and second saccharide-binding agents are used.

Thus, in various embodiments, at least, 5, 10, 15, 25, 30, or 50 or more first saccharide-binding agents are attached to the surface. Preferably, each the first saccharide-binding agents are attached at spatially distinct regions of the substrate. In other embodiments, at least 5, 10, 15, 25, 30, or 50 of more second-saccharide binding agents are used. Preferably, each of the second-saccharide have attached thereto distinguishable labels, i.e., labels that distinguish one-second saccharide-binding agent from another second saccharide-binding agent.

As used herein, a “carbohydrate polymer” includes any molecule with a polysaccharide component. Examples include polysaccharide, a glycoprotein, and glycolipid. While a carbohydrate polymer includes any saccharide molecule containing two or more linked monosaccharide residues, it is understood that in most embodiments, the carbohydrate polymer will include 10, 25, 50, 1000, or 10,000 or more monosaccharide units. If desired, the carbohydrate polymer can be added to the surface after digestion with a saccharide-cleaving agent. Alternatively, the carbohydrate polymer can be added to the surface, allowed to bind to a first saccharide-binding agent attached to the surface, and then digested with a saccharide-cleaving agent.

In general, any agent that binds to a polysaccharide can be used as the first or second saccharide-binding agent. As is known in the art, a number of agents that bind to saccharides have been described. One class of agents is the lectins. Many of these proteins bind specifically to a certain short oligosaccharide sequence. A second class of agents is an antibody that that specifically recognize saccharide structures. A third class of saccharide-binding agent are proteins that bind to carbohydrate residues. For example, glycosidases are enzymes that cleave glycosidic bonds within the saccharide chain. Some glycosidases may recognize certain oligosaccharide sequences specifically. Another class of enzymes are glycosyltransferases, which cleave the saccharide chain, but further transfer a sugar unit to one of the newly created ends.

For the purpose of this application, the term “lectin” also encompasses saccharide-binding proteins from animal species (e.g. “mammalian lectins”). Thus, carbohydrate polymers, like DNA or proteins, clearly have an important biological function which should be studied in greater detail.

A saccharide-binding agent is preferably an essentially sequence-specific agent. As used herein, “Essentially sequence-specific agent” means an agent capable of binding to a saccharide. The binding is usually sequence-specific, i.e., the agent will bind a certain sequence of monosaccharide units only. However, this sequence specificity may not be absolute, as the agent may bind other related sequences (such as monosaccharide sequences wherein one or more of the saccharides have been deleted, changed or inserted). The agent may also bind, in addition to a given sequence of monosaccharides, one or more unrelated sequences, or monosaccharides.

The essentially sequence-specific agent is usually a protein, such as a lectin, a saccharide-specific antibody or a glycosidase or glycosyltransferase.

Examples of saccharide-binding agents lectins include lectins isolated from the following plants: Conavalia ensiformis, Anguilla anguilla, Triticum vulgaris, Datura stramoniuim, Galanthus nivalis, Maackia amurensis, Arachis hypogaea, Sambucus nigra, Erythrina cristagalli, Lens culinaris, Glycine max, Phaseolus vulgaris, Allomyrina dichotoma, Dolichos biflorus, Lotus tetragonolobus, Ulex europaeus, and Ricinus communis.

Other biologically active carbohydrate-binding compounds include cytokines, chemokines and growth factors. These compounds are also considered to be lectins for this patent application.

Examples of glycosidases include α-Galactosidase, β-Galactosidase, N-acetylhexosaminidase, α-Mannosidase, β-Mannosidase, α-Fucosidase, and the like. Some of these enzymes may, depending upon the source of isolation thereof, have a different specificity. The above enzymes are commercially available, e.g., from Oxford Glycosystems Ltd., Abingdon, OX14 IRG, UK, Sigma Chemical Co., St. Lbis, Mo., USA, or Pierce, POB. 117, Rockford, 61105 TJSA.

The saccharide-binding agent can also be a cleaving agent. A “cleaving agent” is an essentially sequence-specific agent that cleaves the saccharide chain at its recognition sequence. Typical cleaving agents are glycosidases, including exo- and endoglycosidases, and glycosyltransferases. However, also chemical reagents capable of cleaving a glycosidic bond may serve as cleaving agents, as long as they are essentially sequence-specific. The term “cleaving agent” or “cleavage agent” is within the context of this specification synonymous with the tern “essentially sequence-specific agent capable of cleaving”.

The cleaving agent may act at a recognition sequence. A “recognition sequence” as used herein is the sequence of monosaccharides recognized by an essentially sequence-specific agent. Recognition sequences usually comprise 2-4 monosaccharide units. An example of a recognition sequence is Galβ1-3 GalNAc, which is recognized by a lectin purified from Arachis hypogaea. Single monosaccharides, when specifically recognized by an essentially sequence-specific agent, may, for the purpose of this disclosure, be defined as recognition sequences.

The reaction conditions for the various essentially sequence-specific agents are known in the art. Alternatively, the skilled person may easily perform a series of tests with each essentially sequence-specific agent, measuring the binding activity thereof, under various reaction conditions. Advantageously, knowledge of reaction conditions under which a certain essentially sequence-specific agent will react, and of conditions under which it remain inactive, may be used to control reactions in which several essentially sequence-specific reagents are present. For example, the second and third sequence-specific reagents may be added to the reaction simultaneously, but via a change in reaction conditions, only the second essentially sequence-specific agent may be allowed to be active. A further change in reaction conditions may then be selected in order to inactivate the second essentially sequence-specific agent and activate the third essentially sequence-specific agent. Some illustrative examples of reaction conditions are listed in the Table 1 below. In addition to the pH and temperature data listed in Table 1, other factor, e.g. the presence of metals such as Zn, or salts of cations such as Mn, Ca, Na, such as sodium chloride salt, may be investigated to find optimum reaction conditions or conditions under which certain essentially sequence-specific agent will be active, while others are inactive.

TABLE 1
Reaction conditions for some essentially sequence-specific agents

| | Condition | | | |
|---|---|---|---|---|
| codes for | serial | | Temp | |
| condition sets | number | pH | (° C.) | Enzyme(s) |
| [symbol] ♥ | 1 | 3.5 | 30 | Jackbean β-galactosidase |
| ♥ | 2 | 5.0 | 37 | Endo a-N |
| | | | | Acetylgalactosidase |
| | | | | α 1,2 Fucosidase |
| | | | | β1,2 galactosidase |
| [symbol] [symbol] | 3 | 5.0 | 25 | Bovine kidney α |
| | | | | Fucosidase |
| ♥ [symbol] | 4 | 7.2 | 25 | Coffee bean α |
| | | | | galactosidase |
| [symbol] ♥ [symbol] | 5 | 5.8 | 55 | B. Fragilis |
| | | | | Endo β-galactosidase |
| | 6 | 6.2 | 25 | Chicken egg lysozyme |
| | 7 | 4.3 | 37 | Bovine testes β 1-3,4,6, |
| | | | | Galactosidase |
| | From | 2-9.5 | 50 | Gly 001-02 |
| | Biodiversa | | | |
| | From | 3.0-8.0 | 50 | Gly 001-04 |
| | Biodiversa | | | |
| | From | 2-11 | 50 | Gly 001-06 |
| | Biodiversa | | | |

Symbols represent enzyme groups which are separable by external conditions.

Diversa Corp. produces Thermophilic Endo/Exo glycosidases with a wide variety of activity in various pH and Temperatures

also possible conditions could be metals and others Zn, Mn, Ca, NaCl

The first saccharide-binding agent may be immobilized using any art-recognized method. For example, immobilization may utilize functional groups of the protein, such as amino, carboxy, hydroxyl, or thiol groups. For instance, a glass support may be functionalized with an epode group by reaction with epoxy silane, as described in the above PCT publication. The epode group reacts with amino groups such as the free ε-amino groups of lysine residues. Another mechanism consists in covering a surface with electrometer materials such as gold, as also described in the PCT publication. As such materials form stable conjugates with thiol groups, a protein may be linked to such materials directly by free thiol groups of cysteine residues. Alternatively, thiol groups may be introduced into the protein by conventional chemistry, or by reaction with a molecule that contains one or more thiol groups and a group reacting with free amino groups, such as the N-hydroxyl succinimidyl ester of cysteine. Also thiol-cleavable cross-linkers, such as dithiobis(succinimidyl propionate) may be reacted with amino groups of a protein. A reduction with sulfhydryl agent will then expose free thiol groups of the cross-linker.

For some applications, it is preferable to design a substrate that contains a plurality of saccharide-binding agents known to bind, or suspected of binding, to a particular carbohydrate polymer of interest. For example, heparin, heparin sulfate, or fragments (such as those produced by heparanase digestion), as well as variant forms of these polysaccharides can be screened for their ability to bind to one or more proteins such as, e.g., aFGF, bFGF, PDGF, VEGF, VEGF-R, HGF, EGF, TGF-beta, MCP-1, -2 and -3, IL-1, -2, -3, -6, -7. -8, -10, and -12, annexin IV, V, and VI, MIP-1 alpha, MIP-1 beta, ecotaxin, thrombospondin, PF-4, IP-10, interferon alpha, interferon gamma, selectin L and selectin P, antithrombin, plasminogen activator, vitronectin, CD44, SOD, lipoprotein lipase, ApoE, fibronectin, and laminin. These putative agents can be attached to a surface (i.e., can be first saccharide binding agents).

In other embodiments, saccharide-binding agents known to or suspect of binding, to a particular carbohydrate polymer can be provided as a second saccharide-binding agent.

The label attached to the second saccharide-binding agent can be any label that is detected, or is capable of being detected. Examples of suitable labels include, e.g., chromogenic label, a radiolabel, a fluorescent label, and a biotinylated label. Thus, the label can be, e.g., colored lectins, fluorescent lectins, biotin-labeled lectins, fluorescent labels, fluorescent antibodies, biotin-labeled antibodies, and enzyme-labeled antibodies. In preferred embodiments, the label is a chromogenic label. The term “chromogenic binding agent” as used herein includes all agents that bind to saccharides and which have a distinct color or otherwise detectable marker, such that following binding to a saccharide, the saccharide acquires the color or other marker. In addition to chemical structures having intrinsic, readily-observable colors in the visible range, other markers used include fluorescent groups, biotin tags, enzymes (that may be used in a reaction that results in the formation of a colored product), magnetic and isotopic markers, and so on. The foregoing list of detectable markers is for illustrative purposes only, and is in no way intended to be limiting or exhaustive. In a similar vein, the term “color” as used herein (e.g. in the context of step (e) of the above described method) also includes any detectable marker.

The label may be attached to the second saccharide-binding agent using methods known in the art. Labels include any detectable group attached to the saccharide or essentially sequence-specific agent that does not interfere with its function. Labels may be enzymes, such as peroxidase and phosphatase. In principle, also enzymes such as glucose oxidase and β-galactosidase could be used. It must then be taken into account that the saccharide may be modified if it contains the monosaccharide units that react with such enzymes. Further labels that may be used include fluorescent labels, such as Fluorescein, Texas Red, Lucifer Yellow, Rhodamine, Nile-red, tetramethyl-rhodamine-5-isothiocyanate, 1,6-diphenyl-1,3,5-hexatriene, cis-Parinaric acid, Phycoerythrin, Allophycocyanin, 4′,6-diamidino-2-phenylindole (DAPI), Hoechst 33258, 2-aminobenzamide, and the like. Further labels include electron dense metals, such as gold, ligands, haptens, such as biotin, radioactive labels.

The second saccharide-binding agent can be detected using enzymatic labels. The detection of enzymatic labels is well known in the art of ELISA and other techniques where enzymatic detection is routinely used. The enzymes are available commercially, e.g., from companies such as Pierce.

In some embodiments, the label is detected using fluorescent labels. Fluorescent labels require an excitation at a certain wavelength and detection at a different wavelength. The methods for fluorescent detection are well known in the art and have been published in many articles and textbooks. A selection of publications on this topic can be found at p. O-124 to O-126 in the 1994 catalog of Pierce. Fluorescent labels are commercially available from Companies such as SIGMA, or the above-noted Pierce catalog.

The second saccharide-binding agent may itself contain a carbohydrate moiety and/or protein. Coupling labels to proteins and sugars are techniques well known in the art. For instance, commercial kits for labeling saccharides with fluorescent or radioactive labels are available from Oxford Glycosystems, Abingdon, UK. Reagents and instructions for their use for labeling proteins are available from the above-noted Pierce catalog.

Coupling is usually carried out by using functional groups, such as hydroxyl, aldehyde, keto, amino, sulfhydryl, carboxylic acid, or the like groups. A number of labels, such as fluorescent labels, are commercially available that react with these groups. In addition, bifunctional cross-linkers that react with the label on one side and with the protein or saccharide on the other may be employed. The use of cross-linkers may be advantageous in order to avoid loss of function of the protein or saccharide.

The label can be detected using methods known in the art. Some detection methods are described in the above-noted WO 93/22678, the disclosure of which is incorporated herein in its entirety. Particularly suitable for the method of the present invention is the CCD detector method, described in the publication. This method may be used in combination with labels that absorb light at certain frequencies, and so block the path of a test light source to the VLSI surface, so that the CCD sensors detect a diminished light quantity in the area where the labeled agent has bound. The method may also be used with fluorescent labels, making use of the fact that such labels absorb light at the excitation frequency. Alternatively, the CCD sensors may be used to detect the emission of the fluorescent label, after excitation. Separation of the emission signal from the excitation light may be achieved either by using sensors with different sensitivities for the different wavelengths, or by temporal resolution, or a combination of both.

In some embodiments, the method further includes acquiring one or more images of the first saccharide-binding agent and the saccharide-binding agent. The information can be is stored, e.g., as a photograph or digitized image. Alternatively, the information provided by the first and second binding image can be stored in a database.

The invention also includes a substrate that includes a plurality of complexes. Each complex includes a first saccharide-binding agent bound to a predetermined location on the substrate. The substrate can also optionally include a saccharide bound to the first saccharide-binding agent and/or a detectable second saccharide-binding agent. In some embodiments, the substrate is provided in the form of a solid support that includes in a pre-defined order a plurality of visual or otherwise detectable markers representative of a saccharide or saccharide sequence or fragment. A preferred substrate is nitrocellulose.

If desired, a substrate containing a plurality of first saccharide-binding agents can be provided in the form of a kit. Diagnostic procedures using the methods of this invention may be performed by diagnostic laboratories, experimental laboratories, practitioners, or private individuals. This invention provides diagnostic kits which can be used in these settings. The presence or absence of a particular carbohydrate polymer, as revealed by its pattern of reacting with saccharide binding agent, may be manifest in a provide sample. The sample can be, e.g., clinical sample obtained from that an individual or other sample.

Each kit preferably includes saccharide-binding agent or agents which renders the procedure specific. The reagent is preferably supplied in a solid form or liquid buffer that is suitable for inventory storage, and later for exchange or addition into the reaction medium when the test is performed. Suitable packaging is provided. The kit may optionally provide additional components that are useful in the procedure. These optional components include buffers, capture reagents, developing reagents, labels, reacting surfaces, means for detection, control samples, instructions, and interpretive information.

The kit may optionally include a detectable second saccharide-binding agent and, if desired, reagents of detecting the second binding agent. The plurality of first saccharide-binding agents is preferably attached at predetermined location on the substrate and a detectable second saccharide-binding agent. In other embodiments, the kit is provided with a substrate and first saccharide-binding agents that can be attached to the substrate, as well as second saccharide-binding agents.

The information provided in the fingerprints described herein can also be used to purify carbohydrate polymers of interest. For example, a carbohydrate polymer can be purified by designing purification schemes based on the saccharide-binding agents to which it binds. In one embodiment, the saccharide-binding agents are provided in column or columns, and a solution containing the carbohydrate polymer is introduced to the column or columns. The carbohydrate polymer of interest is retained on the column or columns. The carbohydrate polymer of interest can then be eluted from the column or columns. In one embodiment, the carbohydrate polymer of interest is using by adding an additional saccharide-binding agent to the column, which binds to, and removes the carbohydrate polymer of interest from the column or columns.

Also within the invention is a method of making a plurality, or library, of carbohydrate polymers that share at least one common function or structural feature, or both. In some embodiments, the carbohydrate polymers are provided as a plurality. If desired, they can be provided on a substrate.

In preferred embodiments the carbohydrate polymers are provided in the form of a focus library, e.g., the members of the focus library are chosen because they bind to a common ligand, or share another common functional or structural property.

For example, in various embodiments, the library of carbohydrate polymers may include variant forms of a polysaccharide such as laminarin, laminarin sulfate, heparin, or heparin sulfate. Members a library based on variant forms of heparin or heparin sulfate polysaccharides can be selected based on the ability of the candidate forms to demonstrate altered properties associated with heparin. For example, the variants may be selected based on their enhanced ability to modulate detachment of the extracellular matrix, to promote cell migration, to bind polypeptides such as chemokines or growth factors, to modulate inflammation, angiogenesis, tumor metastasis, restenosis, or cell proliferation, or to modulate the activity of heparanase. Alternatively, the library may include variant forms of a the carbohydrate polymer moiety of a glycoprotein.

The libraries are constructed by providing a population of carbohydrate polymers. In some embodiments, the population of carbohydrate polymers can be constructed using techniques known in the art for combinatorial chemistry. A carbohydrate fingerprint is generated for one or more members of the population. The member or members of the population are also assayed to determine the degree to which it demonstrates a function or structure of interest. Members of the population containing the desired property are selected for further characterization or modification, if desired. In addition, additional variant carbohydrate polymers can be designed based on the acquired information to result in a population of modified carbohydrate polymers, or a focused library, that have the desired properties.

Fingerprint data generated for the herein described methods may in addition be analyzed using procedures described in U.S. Ser. No. 60/246,009, filed Nov. 3, 2000; and U.S. Ser. No. 60/258,887, filed Nov. 3, 2000, the contents of which are incorporated by reference in their entireties and which are summarized below:

For example, a fingerprint featuring a plurality of addresses, each address containing a numeric value related to binding of a saccharide-binding agent to the carbohydrate polymer, can be analyzed by connecting a first address to at least one other address of the fingerprint to form a map (if the first address is consistent with the at least one other address), determining the map to be internally consistent; and repeating the connecting and determining at least once to form at least one additional map; comparing the map to the at least one additional map to determine if the maps are mutually consistent; and eliminating any mutually inconsistent maps.

Alternatively, or in addition, fingerprint data analysis can be performed using a method for integrating external data to a fingerprint for a sample carbohydrate polymer with the fingerprint featuring a plurality of addresses. Each address contains a numeric value related to binding of a saccharide-binding agent to the sample carbohydrate polymer by converting the external data to form an external fingerprint, and the external data includes at least one assay being performed on a carbohydrate polymer. The external fingerprint is compared to the fingerprint for the sample carbohydrate polymer; and a determination is made for whether the external fingerprint is consistent with the fingerprint for the sample carbohydrate polymer.

Fingerprints for the methods described herein can also be constructed by characterizing the carbohydrate polymer with a fingerprint. The fingerprint may feature a plurality of addresses, each address containing a value obtained from assay data from an experimental assay performed on the carbohydrate polymer. The characterization can include constructing a plurality of maps according to the fingerprint; obtaining additional data for characterizing the carbohydrate polymer; determining if each map is consistent with the additional data; and if the map is not consistent with the additional data, rejecting the map.

In another preferred embodiment, the fingerprints used in the methods described herein can be analyzed by constructing a map for a carbohydrate polymer, where the map includes: characterizing the carbohydrate polymer according to assay data obtained from at least one experimental assay performed on the carbohydrate polymer; decomposing the assay data into a plurality of addresses, each address featuring a value of the assay data; forming a plurality of maps by connecting each address to at least one other address; and transforming each map into a property vector by correlating the value at each address to a feature of the carbohydrate polymer being selected from the group consisting of a function of at least a portion of the carbohydrate polymer, a sequence of at least a portion of the carbohydrate polymer, a structure of at least a portion of the carbohydrate polymer, and a combination thereof.

In another preferred embodiment, the fingerprints used in the methods described herein can be analyzed by constructing a map with a method that includes: providing characterizing data for the carbohydrate polymer; deriving a plurality of maps from the characterizing data; obtaining additional data for characterizing the carbohydrate polymer; determining if the additional data is consistent with each of the plurality of maps; if the additional data is not consistent with a map, eliminating the map; and adding an additional map only if the additional map is consistent with the additional data and with each remaining map.

In another preferred embodiment, the carbohydrate polymers can be characterized with respect to characterizing a sample carbohydrate polymer according to a known carbohydrate polymer having at least one of a known function, a known sequence or a combination thereof. The method includes: performing at least one experimental assay for the sample carbohydrate polymer to obtain assay data; performing an identical experimental assay for the known carbohydrate polymer to obtain comparison assay data; and characterizing the sample carbohydrate polymer according to the known carbohydrate polymer by comparing the assay data to the comparison assay data.

In another preferred embodiment, fingerprints used in the herein described methods are constructed by: providing an experimental assay for determining binding of a saccharide-binding agent to the carbohydrate polymer; detecting whether binding of the saccharide-binding agent to the carbohydrate polymer occurred as raw data; converting the raw data to a numeric value; and placing the numeric value as an address of the fingerprint to form the fingerprint.

In another preferred embodiment, the fingerpreints used in the herein described methods are compared using a method for comparing a plurality of fingerprints for at least a first and a second carbohydrate polymer, each fingerprint featuring a plurality of addresses, each address featuring a numeric value related to binding of a saccharide-binding agent to the carbohydrate polymer. The method includes: comparing the numeric value for at least one address of the fingerprint for the first carbohydrate polymer to the numeric value for the corresponding address of the fingerprint for the second carbohydrate polymer; and determining similarity between the first and second carbohydrate polymers according to the comparison between the numeric values for the addresses.

In another preferred embodiment, the fingerprints are compared using a method for searching through a database of fingerprint data with a fingerprint of a sample carbohydrate polymer, the database containing fingerprint data for a plurality of comparison carbohydrate polymers. The method includes: constructing the database according to an addressing system, the addressing system being at least partially obtained from fingerprint data for the plurality of comparison carbohydrate polymers; converting the fingerprint of the sample carbohydrate polymer to a key; searching through the addressing system with the key; and retrieving fingerprint data from at least one comparison carbohydrate polymer.

In another preferred embodiment, fingerprints are internally analyzed using a method for internally analyzing a fingerprint for extending fingerprint data for a carbohydrate polymer, the fingerprint featuring a plurality of addresses, each address containing a numeric value related to binding of a saccharide-binding agent to the carbohydrate polymer. The method includes: connecting a first address to at least one other address of the fingerprint to form a pattern; if a value for the first address does not contradict a value for the at least one other address, determining the pattern to be internally coherent; and adding each internally coherent pattern to the fingerprint as extended fingerprint data.

In antoher preferred embodiment, the fingerprints are provided by a system for constructing a fingerprint for a sample carbohydrate polymer. The system includes: (a) a wet array, comprising a substrate with a plurality of attached saccharide-binding agents, each saccharide binding agent being located at a predetermined array portion of the wet array, such that the sample carbohydrate polymer is incubated with the wet array to form a complex with a saccharide-binding agent; (b) a detection device for detecting the complex to form raw data; and (c) a conversion module for converting the raw data of each array portion to an address of the fingerprint.

In some preferred embodiments, the fingerprint is generated using a method for constructing a fingerprint for a carbohydrate polymer in a system for constructing a fingerprint for a sample carbohydrate polymer, the system featuring a wet array, the wet array including a substrate with a plurality of attached saccharide-binding agents, each attached saccharide-binding agent being located at a predetermined array portion of the wet array and a detection device. The method includes: incubating the carbohydrate polymer with the wet array under conditions for permitting binding of the carbohydrate polymer to the saccharide-binding agent to occur; detecting whether binding of the saccharide-binding agent to the carbohydrate polymer occurred by the detection device; and adding an address to the fingerprint according to whether binding occurred.

In another preferred embociment, the fingerprints are analyzed in a method for analyzing a sample containing at least one carbohydrate-containing material. The method includes defining a candidate space for determining at least one charateristic of a carbohydrate-containing material in the sample.

General Screening and Diagnostic Methods

Several of the herein disclosed methods relate to comparing carbohydrate polymer fingerprints in cells from a test and reference biological sample. Thus, in its various aspects and embodiments, the invention includes providing a test biological sample which includes at least biological sample that contains, or is suspected of containing, one or more carbohydrate polymers of interest.

Carbohydrate fingerprints for polymers of interest are identified by determining the binding status for one or more saccharide-binding agents for a carbohydrate polymer. Carbohydrate fingerprints of one or more of the carbohydrate polymers in the test biological sample is then compared to carbohydrate fingerprints of carbohydrate polymers from one or more reference biological samples. In various embodiments, the expression of 2, 3, 4, 5, 6, 7, 8, 9, 10, 15, 20, 25, 28, 30, 35, 40, or all of saccharide-binding agents is determined.

The reference biological sample includes one or more carbohydrate polymers from a cell or tissue sample for which the status of the compared parameter is known. The manner in which the carbohydrate fingerprint in the test biological sample reveals the presence, or degree, of the measured parameter depends on the composition of the reference biological sample. For example, if the reference biological sample is derived from cells known to have the parameter of interest, a similar carbohydrate fingerprint in the test biological sample and a reference biological sample indicates the test biological sample has the parameter of interest.

In various embodiments, a carbohydrate polymer in a test biological sample is considered altered if it varies from the corresponding fingerprint in the reference biological sample by more than 1, 2, 3, 5, 10, 15, 20, or 25 saccharide-binding agents.

In some embodiments, the carbohydrate fingerprint of the test biological sample is compared to carbohydrate fingerprints from multiple reference biological samples. The comparison can be made with respect to fingerprints for individual carbohydrate polymers, or to a composite fingerprint that is based on information compiled for multiple polymers.

The test biological sample that is exposed to, i.e., contacted with, the test ligand can be isolated from any number of cells or tissues, i.e., one or more cells, and can be provided in vitro, in vivo, or ex vivo. In various embodiments, the biological sample may be derived from a biological fluid such as, e.g. blood, blood fractions (e.g., serum or plasma), urine, saliva, milk, ductal fluid, tears and semen. Purification of polysaccharides can be performed using methods known in the art.

If desired, the test biological sample can be divided into two or more subpopulations. The subpopulations can be created by dividing a first population of cells, cell extracts, or other carbohydrate-polymer containing fraction, to create subpopulations that are as identical as possible. This will be suitable, in, for example, in vitro or ex vivo screening methods. In some embodiments, various sub-populations can be exposed to a control agent, and/or a test agent, multiple test agents, or, e.g., varying dosages of one or multiple test agents administered together, or in various combinations.

Preferably, the reference biological sample is derived from a tissue type as similar as possible to the test biological sample. In some embodiments, the control biological sample is derived from the same subject as the test sample, e.g., from a distinct region of the subject, or from the same subject taken at a different time (for example, samples can be removed from the subject prior to and after beginning therapy). In other embodiments, the reference biological sample is derived from a plurality of cells. For example, the reference biological sample can be a database of expression patterns from previously tested cells for which one of the herein-described parameters or conditions (e.g., screening, diagnostic, or therapeutic applications) is known.

The subject is preferably a mammal. The mammal can be, e.g., a human, non-human primate, mouse, rat, dog, cat, horse, or cow.

Identifying a Candidate Therapeutic Agent for Treating or Preventing a Pathophysiology Associated with a Carbohydrate Polymer

The methods disclosed herein can also be used to identify candidate therapeutic agents for pathophysiologies associated with a particular carbohydrate polymer fingerprint. The method is based on screening a candidate therapeutic agent to determine if it induces a carbohydrate fingerprint profile in a test biological sample that is characteristic of the carbohydrate fingerprint profile associated with a therapeutic or prophylactic response to the pathophysiology.

In the method, a test biological sample is exposed to a test agent or a combination of test agents (sequentially or consecutively), and the carbohydrate fingerprint of one or more test agents is determined. The carbohydrate fingerprint in the test biological sample is compared to the carbohydrate fingerprint in a reference biological sample. Induction of a carbohydrate fingerprint profile indicative of a therapeutic or prophylactic response to the pathophysiology.

The test agent can be a compound not previously described or can be a previously known compound. An agent effective in effecting a carbohydrate fingerprint of interest, or in suppressing the appearance of a carbohydrate polymer-containing compound, can be further tested for its ability to prevent or ameliorate the pathophysiology, and as a potential therapeutic useful for the treatment of such pathophysiology. Further evaluation of the clinical usefulness of such a compound can be performed using standard methods of evaluating toxicity and clinical effectiveness of therapeutic agents.

Selecting a Carbohydrate Polymer Therapeutic Agent Appropriate for a Particular Subject

Differences in the genetic makeup of individuals can result in differences in their relative abilities to metabolize various drugs. An agent that is metabolized in a subject to act as a carbohydrate polymer therapeutic agent can manifest itself by inducing a change in a carbohydrate fingerprint pattern from that characteristic of a pathophysiologic state to a gene expression pattern characteristic of a non-pathophysiologic state. Accordingly, the carbohydrate fingerprints disclosed herein allow for a putative therapeutic or prophylactic agent suitable for a particular subject to be selected.

To identify an agent that is appropriate for a specific subject, a test biological sample from the subject is exposed to a therapeutic agent, and the carbohydrate fingerprint of one or more carbohydrate polymers is determined. In some embodiments, the test biological sample contains a particular cell type, e.g.; a hepatocyte or an adipocyte. In other embodiments, the agent is first mixed with a cell extract, e.g., an adipose cell extract, which contains enzymes that metabolize drugs into an active form. The activated form of the therapeutic agent can then be mixed with the test biological sample and gene expression measured. Preferably, the biological sample is contacted ex vivo with the agent or activated form of the agent.

The carbohydrate fingerprint in the test biological sample is then compared to the carbohydrate fingerprint of the carbohydrate polymer in a reference biological sample. The reference biological sample is isolated from a cell population or tissue whose pathological status is known. If the reference biological sample is not associated with the pathology, a similar carbohydrate fingerprint profile between the test biological sample and the reference biological sample indicates the agent is suitable for treating the pathophysiology in the subject. In contrast, a difference in expression between sequences in the test biological sample and those in the reference biological sample indicates that the agent is not suitable for treating the pathophysiology in the subject.

If the reference cell is associated with the pathology, a similarity in carbohydrate polymer fingerprint patterns between the test biological sample and the reference biological sample indicates the agent is not suitable for treating the pathophysiology in the subject. A dissimilar gene expression pattern in this instance indicates the agent will be suitable for treating the subject.

Methods and Compositions for Treating Pathophysiology Associated with Variants in a Carbohydrate Polymer in a Subject

Also included in the invention is a method of treating, e.g., inhibiting, preventing or delaying the onset of a pathophysiology associated with a carbohydrate polymer in a subject by administering to the subject an agent which modulates the expression or activity of one or variant of the carbohydrate polymer associated with the pathophysiology. The term “modulates” is meant to include increase or decrease expression or activity of the carbohydrate polymer. Preferably, modulation results in alteration alter the expression or activity of a carbohydrate polymer in a subject to a level similar or identical to a subject not suffering from the pathophysiology. The subject can be, e.g., a human, a rodent such as a mouse or rat, or a dog or cat.

In some embodiments, the agent is an efficacious form of the carbohydrate polymer.

These agents, as well as other polypeptides, antibodies, agonists, and antagonists when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the Therapeutic can be delivered in a controlled release system including, e.g.: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications ofControlled Release 1984. (CRC Press, Bocca Raton, Fla.).

As used herein, the term “therapeutically effective amount means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Cells may also be cultured ex vivo in the presence of therapeutic agents or proteins of the present invention in order to proliferate or to produce a desired effect on or activity in such cells. Treated cells can then be introduced in vivo for therapeutic purposes.

Assessing Efficacy of Treatment of a Pathophysiology

Associated with a Carbohydrate Polymer

Identification of differential fingerprints as described herein also allows for monitoring of the course of treatment of a pathophysiology associated with the carbohydrate polymer. In this method, a test biological sample is provided from a subject undergoing treatment for a pathophysiology associated with the carbohydrate polymer. If desired, test biological samples can be taken from the subject at various time points before, during, or after treatment. One or more carbohydrate fingerprints for one or more carbohydrate polymer is determined. The fingerprints are compared to fingerprints form a reference biological sample which includes cells whose pathophysiologic state is known.

If the reference biological sample is derived from cells that lack the pathophysiology a similarity in the carbohydrate fingerprint between the test biological sample and the reference biological sample indicates that the treatment is efficacious. However, a difference in carbohydrate fingerprints in the test population and this reference biological sample indicates the treatment is not efficacious.

By “efficacious” is meant that the treatment leads to a decrease in the pathophysiology in a subject. When treatment is applied prophylactically, “efficacious” means that the treatment retards or prevents a pathophysiology. Efficaciousness can be determined in association with any known method for treating the particular pathophysiology.

Fingerprint Maps

If desired, fingerprints can be identified and compared using systems and method for characterizing carbohydrate polymers according to maps obtained from experimental data. Preferably, the data is obtained from a plurality of different types of experimental assays for characterizing the carbohydrate polymer. More preferably, at least one such assay involves binding a saccharide-binding agent to the carbohydrate polymer. The map of binding by a plurality of agents is then analyzed in order to at least partially characterize the carbohydrate polymer. The map of binding is used to form a fingerprint, which also incorporates data from other types of assays, for at least a partial characterization of one or more features of the carbohydrate polymer.

These features are preferably derived from maps of the data obtained from assays involving the sample carbohydrate polymer. These maps are more preferably analyzed at a plurality of levels, with each level providing more abstract biological information. Most preferably, new types of experimental data are introduced to the process of analysis at each level, in order to support more complex analyses of the data. Optionally and most preferably, maps are eliminated at each level as being inconsistent with the experimental data. New maps are most preferably added at a higher level only if they are derived from the new experimental data which has been introduced at that level, in order to prevent a combinatorial explosion at successive levels of data analysis.

At a basic level, the analyzed binding data is used to determine a fingerprint for the carbohydrate polymer. This fingerprint is actually a numeric representation of the detection of the presence of binding by the saccharide-binding agents to the carbohydrate polymer. The fingerprint itself thus characterizes the carbohydrate polymer at some level.

Next, the fingerprint is optionally internally analyzed in order to obtain various possible maps which fit the experimental data. For example, certain maps of lectin binding, particularly with sets of model saccharide-binding agents, may be indicative of the presence of a particular type or class of the carbohydrate polymer. Another such map may indicate the presence of a false negative or “hole”, for a lectin or other saccharide-binding agent which should have bound at a particular location, but which did not in fact bind. The presence of a false negative may indicate the presence of a particular type of saccharide “neighborhood”, which affects the binding of the saccharide-binding agent, such that even if a particular sequence is present, binding of the agent itself to the sequence is blocked.

At this level of analysis, optionally many different, mutually contradictory maps may be considered. Preferably, the cut-off or probabilistic threshold for these maps is low, in order to permit as many maps as possible to be considered. These maps are then preferably examined and optionally eliminated in subsequent levels of analysis, as described in greater detail below.

At the next level of analysis, preferably information from other types of assays is incorporated. These assays are optionally and preferably performed with the same or similar experimental material as for the fingerprint data, in order to reduce or even eliminate experimental artifacts. In addition, the use of at least similar experimental material enables results for the sample carbohydrate polymer to be compared to standard, known carbohydrate polymers, without requiring absolute accuracy of the experimental assay, but only reproducibility. For example, the assay could optionally include the use of glycosidases, elimination of reducing ends, and other modifications of the sample carbohydrate polymer. More preferably, previously obtained maps are eliminated at this level as being inconsistent with the experimental data.

The next level preferably enables data to be incorporated from external databases, such that optionally data is used from different experimental materials. Such information could be related to the composition of the saccharide, its source, and possibly other information as well. For example this information could include whether the sample carbohydrate is part of a glycoprotein, the use of other types of carbohydrate binding agents such as cytokines, and so forth. For example, if maps of data obtained from previous stages are definitely incompatible with the source or the composition of the saccharide, then they should be eliminated. The introduction of such data is preferably performed at least partially with information from known carbohydrate-polymers. For example, an unknown saccharide could be classified as “EPO-like”, which could help to guide future experiments.

As further level of analysis, the maps of data should be transformed, such that any reference to the original raw data is eliminated. Such a transformation is preferably performed by locating features of interest within the sample carbohydrate polymer. These features of interest may optionally be short sequences or portions of sequences of monosaccharides within the larger polymer sequence. A very simple example of such a feature is a glycosidase recognition site. Such features may also optionally be described as “sequence-based” features, in that they are characterized by at least a portion of the sequence of the carbohydrate polymer. Such features have the disadvantage of requiring absolute accuracy of the experimental data, rather than mere reproducibility. However, they have the advantage of being comparable over a wide variety of different known carbohydrate polymers, through data obtained from external databases as previously described.

Alternatively and/or additionally and preferably, these features of interest concern functional epitopes and/or sequence-based epitopes having a biological function of interest. By “functional” epitope, it is meant that at least a portion of the carbohydrate polymer appears to be associated with a particular function and/or type of function, regardless of the actual sequence of the carbohydrate polymer. Such a functional epitope may optionally be located through the performance of the same assay on a plurality of carbohydrate polymers, with only the requirement of reproducibility, rather than absolute accuracy. Of course, the functional epitope may also optionally be characterized by a sequence, such that the same epitope may optionally be both a sequence-based epitope and a functional epitope.

Also alternatively and/or additionally and preferably, these features of interest concern “characterization” features. These features are not necessarily discrete portions of the carbohydrate polymer, but rather are indicative of the classification, function or nature of the overall polymer, or some combination thereof. For example, such a characterization feature may enable the carbohydrate polymer to be determined to be “EPO-like”. This determination would not necessarily immediately result in the location of specific functional epitopes within the polymer, for example, but may provide an indication that the carbohydrate polymer should be further examined for the possibility of such functional epitopes being present.

The principles and operation of the present invention may be better understood with reference to the drawings and the accompanying description.

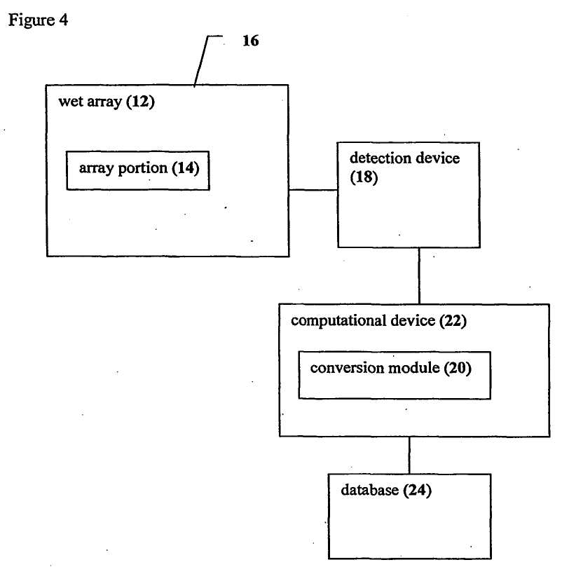

Referring now to the drawings, FIG. 4 shows an exemplary experimental system according to previously incorporated PCT Application No. PCT/IL00/00256 for obtaining the raw data for determining a fingerprint for a carbohydrate polymer of interest. As shown, a system 10 features a wet array 12, in which the actual assay is performed with a plurality of immobilized saccharide-binding agents. Each such immobilized agent is located at a predetermined array portion 14, which is a predetermined location on a substrate 16. Preferably, each array portion 14 features a different immobilized saccharide-binding agent. The plurality of array portions. 14 which are shown compose the entirety of wet array 12. Thus, each array portion 14 is an address on wet array 12; the data obtained from this address forms a part of the fingerprint for the carbohydrate polymer of interest, as described in greater detail below.

The carbohydrate polymer is then incubated with wet array 12, under conditions which permit specific binding of the carbohydrate polymer to one or more immobilized saccharide-binding agents. Such specific binding should result in the formation of a complex between the carbohydrate polymer and the immobilized saccharide-binding agent at a particular array portion 14.

The presence of the complex is then detected by incubating a second, solubilized saccharide-binding agent with wet array 12. The second solubilized agent features a label for detection. Therefore, if the solubilized agent binds to the complex at any particular array portion 14, the presence of such a complex can be detected by detecting the label. A detection device 18 is then used to detect the presence of the label, such that the selection of any particular detection device 18 depends upon the nature of the label. For example, a chromogenic label, such as a dye which becomes excited and fluoresces, can optionally be detected with a camera or other imaging device for detection device 18. Detection device 18 should be able to distinguish between signals from the label from each array portion 14.

Once the signal from each array portion 14 has been collected by detection device 18 and converted to electronic (digital) data, the resultant raw data is preferably transformed to a numeric value for the fingerprint, such that a numeric value for each address of the fingerprint corresponds to an address for wet array 12. The process of transformation is optionally and preferably performed by a conversion module 20, which may be optionally implemented as a software module for operation by a computational device 22. The fingerprint data is then preferably stored in a database 24 which is more preferably also controlled by computational device 22. Of course, a distributed implementation across a network of computational devices is also possible within the scope of the present invention (not shown).

According to preferred embodiments of the present invention, sets of model saccharide-binding agents are used for this assay. The model agents are preferably preselected in order to provide a particular characterization of the sample carbohydrate polymer. For example, the model saccharide-binding agents may optionally be selected in order to be “EPO-like”, for the characterization of the sample carbohydrate polymer according to results which had been previously obtained from EPO. In particular, such model sets of agents should be selected in order to provide data which is particularly indicative of such a characterization. The agents are optionally and more preferably selected by performing experiments with different saccharide-binding agents on known, standard carbohydrate polymers, and then selecting those agents which provide the most useful data for characterization of the sample carbohydrate polymer.

One example of these different types of sets of model agents is a focus library. The members of the focus library are chosen because they bind to a common ligand, or share another common functional or structural property. Examples of the latter include variant forms of glycoproteins such as EPO, interferon alpha, CGSF, and HCG.

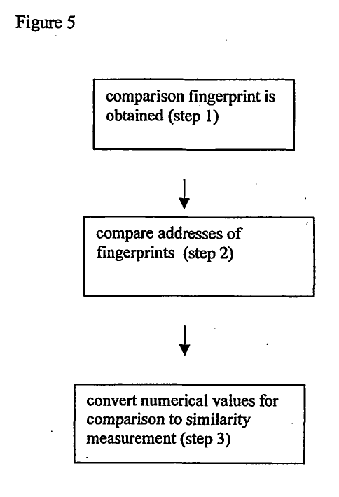

Next, optionally and preferably, a comparison method is performed for comparing the fingerprint of the sample carbohydrate polymer to at least one other fingerprint. More preferably, the fingerprint for comparison is obtained from a standard, known carbohydrate polymer, although alternatively, the other fingerprint could also optionally be obtained from another sample carbohydrate polymer. An example of such a method is described with regard to FIG. 5.

In step 1, the comparison fingerprint is obtained. As previously described, the comparison fingerprint is preferably obtained from a standard known carbohydrate polymer. Regardless of the source of the fingerprint data, however, preferably the comparison fingerprint data includes information about the experimental conditions, including at least the set of saccharide-binding agents which were used to obtain the data, and more preferably including such information as washing conditions, stringency of the incubation conditions, the type of label on the solubilized saccharide-binding agent, and so forth.

In step 2, the actual address(es) of the fingerprints are compared. Optionally, the comparison is performed address by address, with at least a positive result of the comparison being given a positive numerical value. More preferably, a negative result of the comparison is given a negative numerical value. Step 2 is then preferably repeated for all addresses which are to be compared.

In step 3, the total numerical values for the address-by-address comparison are preferably converted to a similarity factor according to some function. The function is optionally simple, for example by adding all of the positive and negative values from the address-by-address comparison process. Alternatively and preferably, the results can be weighted. More preferably, the results are weighted according to the previously described interpretive information from the experimental conditions, such that a greater weight could optionally be given to the result of a comparison between two addresses of the fingerprints in which more certainty can be assigned to the experimental result, for example.

An example of a quantitative tool for comparing two fingerprints optionally and more preferably employs phylogenetic analysis, which has the advantage of returning a distance between two or more fingerprints, as opposed to a simple numeric measurement of similarity/dissimilarity. Originally used for examining evolutionary relationships between biological sequences, such as protein or DNA sequences for example, phylogenetic analysis provides a quantitative measure of the distance, or the degree of difference between two or more sequences. The use of phylogenetic analysis is particularly preferred for the optional but preferred embodiment of the present invention, in which the fingerprint of the sample carbohydrate polymer is compared to a database containing a plurality of such fingerprints. More preferably, the fingerprint data is for standard carbohydrate polymers. In any case, for this preferred embodiment of the present invention, step 3 is replaced by a different function, which optionally requires step 2 to be repeated for each fingerprint in the database.

Since phylogenetic analysis has been investigated for many years, and is a well-known topic in the art, many different methods are known in the art. In addition, a variety of companies offer a variety of products and utilities for analyzing phylogenetic information.

According to the present invention, optionally and more preferably, the following function is used for calculating phylogenetic information, in which the information of the fingerprints is expressed as a matrix of distances. These distances are optionally obtained according to some known function, such as a Hamming function, for example. According to a preferred embodiment of the invention, the distances are obtained as follows:

$$D = {\sum }_{i = 1}^{N} {\sum }_{j = 1}^{C} V i \qquad ( 4 )$$

Where:

D is the expression for the distance;

N is the number of addresses in fingerprint1 and fingerprint2;

C is the maximum number of colors that can be distinguished in address i of the fingerprints;

Vi is 1 if a color that found in address i of fingerprint1 exists in the same address i infingerprint2, otherwise Vi is zero.

The previous two Figures described some basic tools for obtaining experimental fingerprint data, and for comparing fingerprint data between two or more carbohydrate polymers. The next Figures describe methods for deriving higher level information from the fingerprint data, such as maps which characterize the sample carbohydrate polymer, for example. The method of each subsequent Figure enables increasing higher levels of information to obtained, and also optionally allows maps or other characterizations of the sample carbohydrate polymer which do not fit the experimental data to be eliminated. Preferably, at each higher level, additional experimental data and analyses are incorporated into the process for obtaining and examining the maps, in order to characterize the sample carbohydrate polymer as much as possible, and also in order extend the useful information which can be derived from individual experiments.

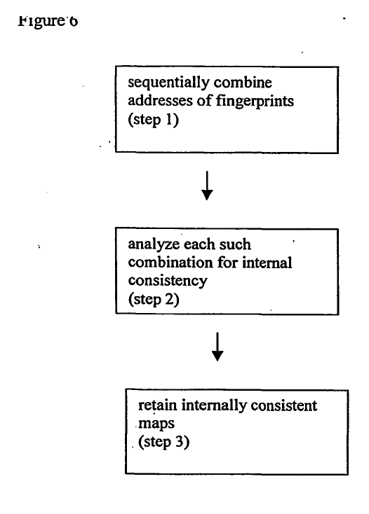

According to preferred embodiments of the present invention, the fingerprint of the sample carbohydrate polymer is itself internally analyzed in order to extend the fingerprint data, as described with regard to the method of FIG. 6. According to this exemplary method, the fingerprint addresses are first recursively analyzed in order to find simple maps, or map fragments. Next, these map fragments are assembled to larger maps, again preferably through a recursive analysis. Optionally and more preferably, the maps are transformed into property vectors, or property descriptors, for use in QSAR (quantitative structure-activity relationship) algorithms. This translates the fingerprint data into a set of numbers directly describing structural properties (i.e., the level of sialic acid content, the existence or absence of certain monomers or dimers, and so forth). QSAR can in turn optionally be used for activity prediction in molecular drug design.

As shown with regard to FIG. 6, in the first stage of the method, a first set of maps which characterize the sample carbohydrate is preferably created, optionally through recursive analysis of the fingerprint data. Such a recursive analysis may optionally simply take the form of sequentially combining each address of the fingerprint with a sequence of one or more other addresses in step 1. Next, in step 2, each such combination is analyzed in order to determine if the map (or map fragment) is internally coherent. In step 3, those maps or map fragments which have been shown to be internally coherent are retained for the next level of analysis.

As an example for this type of analysis, a map may obtained from an experiment in which the sample carbohydrate polymer is first digested with a cleaving agent, and in subsequent steps reacted with binding agents. Such an assay is described in more detail with regard to PCT Application No. PCT/IL00/00256. However, as a brief example, a sample carbohydrate polymer which is labeled at the reducing end is reacted with a first saccharide-binding agent, which may optionally be a glycosidase with the recognition sequence a. In a control reaction, the labeled sample carbohydrate polymer is left untreated. The reactions are then independently further reacted with an immobilized saccharide-binding agent, which may optionally be a lectin with the recognition sequence b. After washing off unbound sample carbohydrate polymer, a detection step is carried out. The presence of the label indicates that site b is present in the sample carbohydrate polymer.

By comparing reactions where the first saccharide-binding agent is present, with independent control reactions where the first saccharide-binding agent is absent, the effect of the glycosidase on the presence of the label can be determined. For instance, if the label is detected in the control reaction after binding to the lectin with recognition sequence b, but not in a reaction where the first saccharide-binding agent is a glycosidase with the recognition sequence a, the sequence of recognition sites is b-a-reducing end. On the other hand, if the label is present in both control and glycosidase reactions, this indicates that the sequence of recognition sites is a-b-reducing end. The recognition site a may not be located inside the sample carbohydrate polymer, i.e., may not exist in the saccharide sequence.

According to preferred embodiments of the present invention, step 1 is performed by first placing each address of the fingerprint as a node on a hierarchical tree. Depending upon the type of data that is represented by the fingerprint address, the address may optionally appear on more than one node. Preferably, the hierarchy of the tree is constructed according to a plurality of categories of data. For example, part of the tree may optionally represent simple binding of the saccharide-binding agent to the sample carbohydrate polymer. This part of the tree would then be preferably structured according to characterization of each saccharide-binding agent, for example according to the type of agent (lectins, antibodies, etc.), the effect of the agent on the sample carbohydrate polymer (binding, cleavage, etc.), the type of label for the solubilized saccharide-binding agent.

Next, in step 2, the tree can be recursively examined by using each address of the tree as the root node, for example, or alternatively by traveling from each node of the tree to the other nodes of the tree to establish the map or map fragments. The advantage of this method is that if the tree is constructed according to biologically useful categories and/or parameters, the maps which are constructed from the nodes of the tree should be internally coherent. This process may optionally be repeated a number of times in order to construct larger maps.

An example of a procedure for constructing and examining such trees is optionally and preferably performed as follows. Lectins can optionally be used as the saccharide-binding agents for the experimental assay, such as the assay described with regard to FIG. 4. Preferably, such lectins are used as pairs of lectins: a first lectin for being immobilized to the surface of the solid support, to which the carbohydrate polymer initially binds to form a complex; and a second solubilized lectin for binding to the complex. The second lectin preferably features a label in order to permit the presence of the complex to be detected. These pairs of lectins can optionally be correlated with a clustering algorithm, such that the “relatedness” or distance between results for pairs of lectins can be determined from their binding behavior to the carbohydrate polymer. Such correlations can then optionally be used to form the tree, such that each node of the tree is related to other nodes according to the relative distance. Alternatively, the correlation can optionally be used in order to structure the nodes of the tree according to the behavior of the lectins with regard to a standard, known carbohydrate polymer.

One example of a measurement according to which the lectins could be organized in the tree is the Hamming distance, as previously described, or the Jaccard similarity measure. The Jaccard similarity measure between non-zero vectors v₁ and v₂ is defined as follows:

Jaccard measure=a₁₁/(a₁₁+a₀₁+a₁₀)

where a₁₁ is the number of dimensions in which v₁ has the value i and v₂ has the value j. This similarity measure can be used to determine the similarity of results between pairs of lectins, as well as the similarity of results between different fingerprints. For example, the tree could optionally be constructed from different fingerprints for known carbohydrate polymers, which would then be examined for their similarity to the results for the sample carbohydrate polymer.

Preferably, multiple types of fingerprint data are incorporated into these maps, optionally also including fingerprint data which involves the modification of the sample carbohydrate polymer before the assay is performed. For example, the polymer could optionally be modified with glycosidases for cleaving the molecule; elimination of reducing ends; and with glycosyltransferases for adding one or more saccharides, optionally with a label, to the sample carbohydrate polymer. Modification with saccharide(s) having a label is particularly preferred for “double-label” experiments, in which the second saccharide-binding agent of the assay of FIG. 4 would have the second label. The map of the two labels would thus provide additional information concerning the structure of the sample carbohydrate polymer.

It should be noted that these different types of experimental data may optionally be incorporated into a single fingerprint for the sample carbohydrate polymer, although such incorporation is not necessary. Alternatively, the different types of data may be used as an adjunct to the fingerprint for creating the maps for the polymer. In any case, these different types of experimental data should be obtained from experimental assays performed on at least similar experimental material, with at least similar conditions. More preferably, the experimental material and conditions are identical, particularly for comparisons between different polymers, such as between a standard, known carbohydrate polymer and the sample carbohydrate polymer.

Optionally and more preferably, the maps are transformed into property vectors, or property descriptors, for use in QSAR (quantitative structure-activity relationship) algorithms, for example. Each property vector is a quantitative description of structural properties and/or features of the sample carbohydrate polymer. Each numeric value in the vector preferably corresponds to a particular property or feature, such as the level of sialic acid content, the existence or absence of certain monomers or dimers in the carbohydrate sequence, and so forth). Such a property vector could also optionally feature data for describing more qualitative properties.

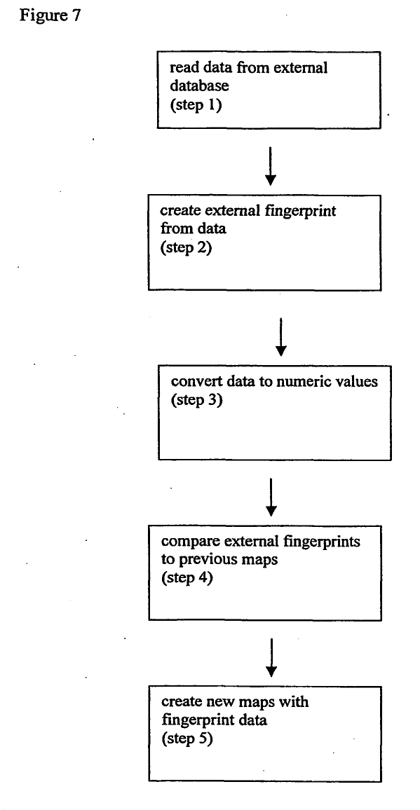

The process of translation is preferably performed by correlating a plurality of numeric values of the fingerprint in order to build the map. Such a correlation is optionally performed by comparing the fingerprint data to a “template”, in order to determine if the property or feature exists. Alternatively, the value in the property vector could optionally be obtained by integrating results from other types of experiments, as described in greater detail with regard to FIG. 7 below. For example, the value in the property vector could optionally be derived from the saccharide content of the sample carbohydrate polymer.

Such additional information may enhance the data interpretation in a number of respects. First, it can optionally be used to eliminate impossible or at least highly improbable recognition sites from those sites which have determined to be possible sites from the different types of experimental assays. For example, for assays in which lectins are used as a saccharide-binding agent, many lectins specifically bind to both glucose (Glc) and mannose (Man), yet many glycans do not contain Glc. Thus, the presence of binding to these lectins indicates the presence of Man alone.

In addition, such information can optionally suggest ambiguities in data interpretation, and add information that is not present in the data. An example of the latter function would be the detection of the presence of Kdo, which is a monosaccharide in LPS (lipopolysaccharides), yet may not be detected according to lectin binding data. Such information may also present a strong clue to confirm/reject certain hypotheses.

Such information should not be limited to monosaccharide composition, however, as this is only intended as a non-limiting illustrative example. Instead, this information may optionally include data from experimental assays; structural information, such as how many length species are created by a certain cleavage of a polymer; medical and origin information, since for example mammalian carbohydrate polymers are more limited in monosaccharide composition then plant carbohydrate polymers, and both are more limited than bacterial carbohydrate polymers.

FIG. 7 is a flowchart of an exemplary method according to the present invention for extending the fingerprint data by integration of data from external databases. By “external databases”, it is meant that the data is obtained from experiments which are not performed on the same material, such that the same experimental conditions do not necessarily apply to both sets of data. Such information could be related to the composition of the saccharide, its source, and possibly other information as well.

For example, this information could include whether the sample carbohydrate is part of a glycoprotein, the use of other types of carbohydrate binding agents such as cytokines, and so forth. The introduction of such data is preferably performed at least partially with information from known carbohydrate polymers, such as EPO, for example, as a standard, reference carbohydrate polymer.

As shown with regard to FIG. 7, in step 1, the data is read from the external database, and the format of the data is analyzed. In step 2, if the format of the data includes one or more numerical values which characterize specific aspects of the polymer, then these values are optionally used to create a “fingerprint” for the sample carbohydrate polymer. For example, if an assay has been performed with the sample carbohydrate polymer to determine the saccharide content, then the relative amounts and identity of the different types of saccharides are clearly convertible to a fingerprint of such data.

Alternatively, in step 3, if the format of the data includes raw experimental results, such as a map of bands on a PAGE (polyacrylamide gel electrophoresis) gel after cleavage of the carbohydrate polymer with a glycosidase for example, then the data is preferably converted to one or more numeric values. For example, the map of bands could optionally be converted by determining the presence or absence of a band at a particular molecular weight, and then creating a “fingerprint” with binary values (positive/negative) at each molecular weight. Alternatively, the fingerprint could optionally include the series of molecular weights for the bands as a sequence of numerical values. It should be noted that PAGE gel assays are intended only as a non-limiting example, and that other types of assay data could also optionally be incorporated, such as column chromatographic data for example.

The format of the data may also optionally include two different types of experimental results, which would then preferably be correlated in order to form the fingerprint. For example, the PAGE gel assay could be performed with the addition of end-labeling with various types of glycosyltransferases or other end-labeling mechanisms. The gel would then contain two types of data: the presence of bands at specific molecular weights; and the presence of specific labeled bands. The fingerprint could then optionally be created to indicate both types of data as numeric values, for example as the molecular weight of the bands with binary (positive/negative) values for indicating the effect of labeling.

Preferably these external “fingerprints” are also created for standard known carbohydrate polymers as references for comparison to the data for the sample carbohydrate polymer. Such external “fingerprints” could optionally be derived by the performance of specific experimental assays on the standard carbohydrate polymer, or alternatively could be derived by converting existing data to the fingerprint format.

In step 4, these fingerprints are preferably compared to the maps which were derived for the sample carbohydrate polymer from the previous level in FIG. 6. If any of these maps are inconsistent with the additional fingerprint data, they are optionally and preferably eliminated. For example, lectin binding information may indicate the possibility that the monosaccharide Fuc (fucose) is absent. On the other hand, such a possibility may be directly contradicted by the monosaccharide composition of the carbohydrate polymer, which may indicate the presence of Fuc. In such a situation, the addition of the latter data may optionally indicate that a map which does not include Fuc should preferably be eliminated as being inconsistent with the additional data.

In step 5, optionally and more preferably, the additional fingerprint data is used to create new maps. These new maps are most preferably created according to the method of FIG. 6, which is suitable for use with fingerprint data of this format, regardless of the source of the experimental data.

Both the optional creation of new maps and the optional elimination of existing maps are examples of the examination of the probability space for the carbohydrate polymer. Unlike for the method described below, these maps may still optionally be directly related to the fingerprint or other experimental data. However, the probability space is more difficult to search than for other types of biological polymers, such as DNA for example, since there is no requirement for accuracy of the experimental data, but only for reproducibility. Thus, the probability or combinatorial space is increased even beyond that which is searched for other types of biological polymers.

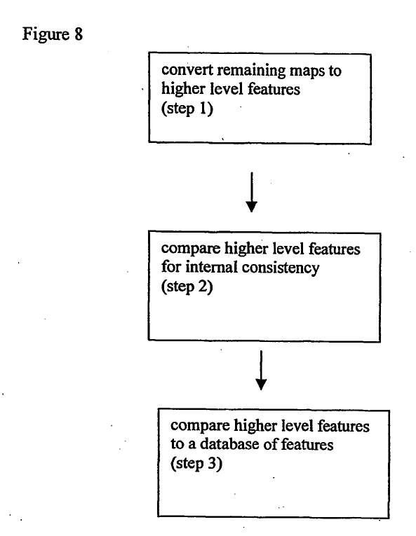

FIG. 8 is a flowchart of an exemplary method according to the present invention for locating features of interest within the sample carbohydrate polymer, By this point, the maps should no longer include any reference to the original raw data, but instead should be composed of sequences of elements. Some raw data may not yield any useful information. The sequences of elements can now be compared to a three-dimensional database, which stores pieces of three-dimensional (structural) information.

This process is actually a combinatorial search, or a search in combinatorial space, since each of the maps represents a possible combination of related elements for describing the sequence, structure, function, or some combination thereof, of the carbohydrate polymer. These maps can in turn be used to search for different higher level features of the carbohydrate polymer, which are related to particular sequences, structures and/or functions of interest within the polymer.

As shown with regard to FIG. 8, in step 1, the remaining maps are first converted to higher level features, if necessary (this step may optionally have already been performed as part of the process of creating the maps). For example, the maps are preferably converted to conform to various functional epitopes and/or sequence-based features, as well as to characterization features. This step is particularly aided by the presence of data from previous comparisons to standard reference carbohydrate polymers, since such comparisons are particularly useful for locating functional features.

These features of interest may optionally be short sequences or portions of sequences of monosaccharides within the larger polymer sequence. A very simple example of such a feature is a glycosidase recognition site. Such features may also optionally be described as “sequence-based” features, in that they are characterized by at least a portion of the sequence of the carbohydrate polymer. Such features have the disadvantage of requiring absolute accuracy of the experimental data, rather than mere reproducibility. However, they have the advantage of being comparable over a wide variety of different known carbohydrate polymers, through data obtained from external databases as previously described.

Alternatively and/or additionally and preferably, these features of interest concern functional epitopes and/or sequence-based epitopes having a biological function of interest. By “functional” epitope, it is meant that at least a portion of the carbohydrate polymer appears to be associated with a particular function and/or type of function, regardless of the actual sequence of the carbohydrate polymer. Such a functional epitope may optionally be located through the performance of the same assay on a plurality of carbohydrate polymers, with only the requirement of reproducibility, rather than absolute accuracy. Of course, the functional epitope may also optionally be characterized by a sequence, such that the same epitope may optionally be both a sequence-based epitope and a functional epitope.

Also alternatively and/or additionally and preferably, these features of interest concern “characterization” features. These features are not necessarily discrete portions of the carbohydrate polymer, but rather are indicative of the classification, function or nature of the overall polymer, or some combination thereof. For example, such a characterization feature may enable the carbohydrate polymer to be determined to be “EPO-like”. This determination would not necessarily immediately result in the location of specific functional epitopes within the polymer, for example, but may provide an indication that the carbohydrate polymer should be further examined for the possibility of such functional epitopes being present.

In step 2, these higher level features are compared for internal consistency. If any two such features are inconsistent or mutually exclusive, then optionally and preferably, both such features are removed from further consideration, as it is not possible to determine which is correct. However, if further data becomes available, then alternatively one of the features could be retained, according to the data, for example as previously described.

In step 3, the higher level features are compared to a database of such features, which is preferably embodied as a three-dimensional database containing structural and/or functional components of carbohydrate polymers. For example, such a feature could optionally be used to locate an epitope of interest, which could then provide information concerning the type or function of the sample carbohydrate polymer.

The invention will be further illustrated in the following examples, which do not limit the scope of the appended claims.

### EXAMPLE 1

### Glycomolecule Analysis Using Antibodies as First and Second Sequence-Specific Agents

This example further illustrates the technique of analyzing glycomolecules according to the invention. As a first and second sequence-specific agent, antibodies are used. The following tables lists the results of reactions with two different saccharides denoted for purposes of illustration, HS and NS.

The structure of the sugars is as follows:

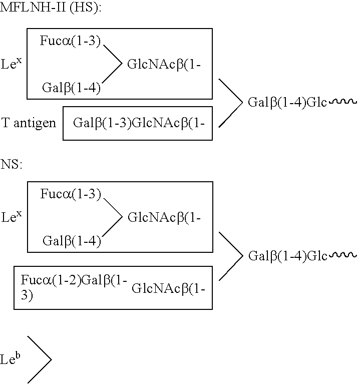

Table 2 lists the results of the reaction between the saccharide and the first and second essentially sequence-specific agents, which are antibodies against T-antigen, Lewis^{x} (Le^{x}), or Lewis^{b} antigen (Le^{b}). The first essentially sequence-specific agent is immobilized on a matrix, preferably a solid phase microparticle. The second essentially sequence-specific agent is labeled with a fluorescent agent, i.e., nile-red or green color. In addition, the reducing end of the saccharide is labeled, using a label clearly distinguishable from the nile-red or green color label which act as markers for the second essentially sequence-specific agents. Table 2 lists the reactions for the saccharide HS, while table 3 lists the reactions for the saccharide NS.

TABLE 2

| On the matrix | anti T-antigen | anti-Le^{X} | anti-Le^{b} |
|---|---|---|---|
| Saccharide bound | HS | HS | |
| Second mAb | nile-red anti-Le^{X} | | |
| Signal | nile-red, reducing | Reducing end | none |
| | end | | |

TABLE 3

| On the matrix | anti T-antigen | anti-Le^{X} | anti-Le^{b} |
|---|---|---|---|
| Saccharide bound | | NS | NS |
| Second mAb | | Green anti-Le^{b} | nile-red anti- |
| | | | Le^{X} |
| Signal | | Green, reducing | nile-red, |
| | | end | reducing end |

In summary, the following signals are now detectable in the reactions of the saccharide HS or NS (rows) when using the indicated antibodies as first essentially sequence-specific agent (columns):

TABLE 4

| On the | | | |
|---|---|---|---|
| matrix | anti T-antigen | anti-Le^{X} | anti-Le^{b} |
| HS | nile-red, reducing | Reducing end | |
| | end | | |
| NS | | Green, reducing | nile-red, reducing |
| | | end | end |
| NS | | Green, reducing | nile red, reducing |
| | | end | end |

After the label has been detected and the result recorded for each reaction, a third essentially sequence-specific agent is added. In this example, two independent reactions with a third essentially sequence-specific agent are used. The solid phase carrying the sugar molecule may now be advantageously divided into aliquots, for reaction with either α1-2 Fucosidase or Exo β galactosidase (third essentially sequence-specific agents). Alternatively, three sets of reactions with a first and second essentially sequence-specific agent may be carried out.

TABLE 5
reactions after applying α1-3,4 Fucosidase:

| On the matrix | anti T-antigen | anti-Le^{X} | anti-Le^{b} |
|---|---|---|---|
| HS | reducing end | | |
| NS | | | |

TABLE 6
reaction after applying Exo β galactosidase from
D. pneumoniae (EC 3.2.1.23 catalog number 1088718 from
Boehringer Mannheim, 68298 Mannheim, Germany)

| On the matrix | anti T-antigen | anti-Le^{X} | anti-Le^{b} |
|---|---|---|---|
| HS | nile-red | | |
| NS | | Green | nile-red |

TABLE 7
reactions after applying α1-2 Fucosidase:

| On the matrix | anti T-antigen | anti-Le^{X} | anti-Le^{b} |
|---|---|---|---|
| HS | nile-red, reducing | Reducing end | |
| | end | | |
| NS | | Reducing end | |

From the data gathered as explained above, a glycomolecule identity (GMID) card can now be created. An example for such information is listed in Table 8 for saccharide HS and in Table 9 for saccharide NS.

TABLE 8

| On the matrix | anti T-antigen | anti-Le^{x} | Anti-Le^{b} |
|---|---|---|---|
| 0 | nile-red, reducing | Reducing end | |
| | end | | |
| 1 | reducing end | — | — |
| 2 | nile-red | | |
| 3 | nile-red, reducing | Reducing end | |
| | end | | |

TABLE 9

| On the matrix | anti T-antigen | anti-Le^{X} | anti-Le^{b} |
|---|---|---|---|
| 0 | | Green, reducing | nile red, reducing |
| | | end | end |
| 1 | — | — | — |
| 2 | | Green | nile red |
| 3 | | Reducing end | |

The identity of the second and third essentially sequence-specific agents need not be disclosed in such a data list For the purpose of comparison, it is sufficient that a certain code number (1, 2 or 3 in the above tables) always identifies a certain combination of reagents.

### EXAMPLE 2

A Scheme for the Sequential Labeling of Reducing Ends

As has been indicated in the description and example above, the method of the invention advantageously uses labeling of the saccharide to be investigated at its reducing end. However, this labeling technique may be extended to sites within the saccharide, and thus contribute to the method of the invention, by providing more information. As it is possible to label the saccharide within the chain, by cleavage using an endoglycosidase followed by labeling of the reducing end, it is therefore possible to obtain a labeled reducing end within the saccharide chain. As that reducing end is necessarily closer to the binding sites for the first, second and third essentially sequence-specific agents, compared to the original reducing end, the use of an internally created labeled reducing end provides additional information. Moreover, it is possible, by sequentially labeling of reducing ends according to the method described further below, to identify the sites for distinct glycosidases in sequential order on the chain of the saccharide to be investigated.

The method of sequential labeling of reducing ends is now described in more detail in the following steps:

1. Blocking:

A polysaccharide having a reducing end is incubated in a solution containing NaBH₄/NaOH at pH 11.5.

This treatment blocks the reducing end, so that the polysaccharide is now devoid of a reducing end (RE).

2. Exposing:

The polysaccharide of step 1 is treated with an endoglycosidase. If the recognition site for that endoglycosidase is present within the polysaccharide, a new reducing end will be created by cleavage of the polysaccharide. The solution now contains two saccharides: the fragment with the newly exposed RE in the endoglycosidase site, and the second fragment whose RE is blocked.

3: Labeling of the Reducing End

This reaction may be carried out using e.g., 2-aminobenzamide (commercially available in kit form for labeling saccharides by Oxford Glycosystems Inc., 1994 catalog, p. 62). After the reaction under conditions of high concentrations of hydrogen and in high temperature (H+/T), followed by reduction, has been completed, the mixture contains two fragments, one of which is labeled at its reducing end, while the other remains unlabeled due to the fact that its reducing end is blocked.

Another way to label reducing ends is by reductive amination. Fluorescent compounds containing arylamine groups are reacted with the aldehyde functionality of the reducing end. The resulting CH═N double bond is then reduced to a CH₂—N single bond, e.g., using sodium borohydride. This technology is part of the FACE (Fluorophore assisted Carbohydrate Electrophoresis) kit available from Glyko Inc., Novato, Calif., USA, as detailed e.g., in the Glyko, Inc. catalog, p. 8-13, which is incorporated herein by reference.

4. Reaction with a Second Endoglycosidase

A second endoglycosidase may now be reacted with the saccharide mixture. The new reaction mixture has now three fragments, one with an intact reducing end, a second with a reducing end labeled by 2-aminobenzimide, and a third with a blocked reducing end.

### EXAMPLE 3

### Derivation of Structural Information from a Series of Reactions with Essentially Sequence-Specific Agents

This example further illustrates the method of the invention, i.e., the generation of data related to the structure of the saccharide by using a set of reactions as described further above. The example further demonstrates that sequence information can be deduced from the set of reactions.

In some cases, the reagents used may not react exactly as predicted from published data, e.g. taken from catalogs. For instance, the lectin Datura stramonium agglutinin as described further below is listed in the Sigma catalog as binding GlcNac. However, in the reactions detailed further below, DSA is shown to bind to Coumarin 120-derivatized Glc (Glc-AMC). It appears that Glc-AMC acts like GlcNac for all purposes, because of the structural similarity between these compounds. Further, as apparent from the results below, the endogalactosidase used cleaves not only at galactose residues, but also the bond connecting the Glc-AMC group to the rest of the saccharide.

It is apparent that the essentially sequence-specific agents used in the practice of the invention may in some cases have fine specificities that vary from the specificity of these agents given in published material, e.g., catalogs. Such reactions can quickly be identified by using the method of the invention with saccharides of known structure. The results found may then be compared with expected results, and the differences will allow the identification of variant specificities of the essentially sequence-specific agents used. Such variation from published data in fine specificities of essentially sequence-specific agents may then be stored for future analysis of unknown saccharides structures using these agents.

In the following, the method of the invention is illustrated using an end-labeled pentasaccharide and various lectins and glycosidases. The pentasaccharide has the structure Gal-β(1,4)[Fuc-α(1,3)]-GlcNAc-β(1,3)-Galβ(1,4)-Glc. The pentasaccharide is branched at The GlcNAc position having fucose and galactose bound to it in positions 3 and 4 respectively. The pentasaccharide is labeled at its reducing end (Glc) with Coumarin-120 (7-amino-4-methyl coumarin, available, e.g., from Sigma, catalog No. A 9891). The coupling reaction may be carried out as described above for the labeling of reducing ends by using arylamine functionalities. Coumarin-120, when excited at 312 nm emits blue fluorescence. As first and second essentially sequence-specific agents, Endo-β-Galactosidase (EG, Boehringer Mannheim) and Exo-1,3-Fucosidase (FD, New England Biolabs) are used. The reaction conditions for both reagents are as described in the NEB catalogue for Exo-1,3-Fucosidase.

Three reactions were carried out The first included Fucosidase (FD) and Endo-Galactosidase (EG), the second, FD only, and the third, EG only. A fourth reaction devoid of enzyme served as control.

In order to ascertain that the enzymes had digested the saccharide, the various reactions are size-separated using thin-layer chromatography (TLC).

After separation, the saccharides on the TLC plate may detected by exposing the plate to ultraviolet light. The results are shown in the following illustration.

[image]

In reaction 4, no glycosidase was added, so the saccharide is intact and moves only a small distance on the plate. The fragment of reaction 2 is second in molecular weight, while the fragments of reactions 1 and 3 appear to be equal. From these data, it can be concluded that the sequence of the glycosidase sites on the saccharide is FD-EG—reducing end (coumarin-label).

The above pentasaccharide is now tested by a set of reactions as described further above. As first and second essentially sequence-specific agents, lectins were used. The lectins (Anguilla Anguilla agglutinin (AAA), catalog No. L4141, Arachis Hypogaea agglutinin (PNA), catalog No. L0881, Ricinus communis agglutinin (RCA I) catalog No. L9138, Lens Culinaris agglutinin (LCA) catalog No. L9267, Arbus Precatorius agglutinin, (APA) catalog No. L9758) are available from Sigma. Lectins are also available from other companies. For instance, RCA I may be obtained from Pierce, catalog No. 39913. Lectins are immobilized by blotting onto nitrocellulose filters.

The reaction buffer is phosphate-buffered saline (PBS) with 1 mM CaCl and 1 mM MgCl. After binding of the lectins, the filter was blocked with 1% BSA in reaction buffer. As controls, reactions without lectin and with 10 μg BSA as immobilized protein were used.

The results of the reactions are indicated in Table 10. A plus indicates the presence of 312 nm fluorescence, which indicates the presence of the coumarin-labeled reducing end. The numerals 1-4 in the table indicate reactions as defined above.

TABLE 10

| | AAA | PNA | LCA | DSA | RCA I |
|---|---|---|---|---|---|
| 1 | | | | ++ | |
| 2 | | ++ | | ++ | ++ |
| 3 | | | | ++ | |
| 4 | ++ | ++ | | ++ | ++ |

From the results as listed in Table 10 (reaction 4-control) it is evident that lectins AAA, PNA, DSA and RCA-I bind the saccharide. Therefore, Fucose, Gal(1-3)GlcNAc, GlcNAc, and Galactose/GalNAc must be present in the saccharide, as these are the respective saccharide structures that are recognized by AAA, PNA, DSA and RCA-I. It is further evident that the above described glycosidases Fucosidase and Endo-βGalactosidase recognize cleavage sequences in the saccharide. These sequences are Fuc (1-3/1-4) GlcNAc and GlcNAcβ(1-3)Galβ(1-3/4)Glc/GlcNAc, respectively.

It can further be deduced that both glycosidase sites are located between the fucose sugar and the reducing end, as the end is cleaved by either glycosidase when AAA (which binds to fucose) is used as immobilized lectin. The reaction with DSA, on the other hand, allows the deduction that either the GlcNAc monosaccharide is located between the glycosidase sites and the reducing end, or that Glc is directly bound to the coumarin, as neither glycosidase cleaves off the reducing end when DSA is used as immobilized agent.

Moreover, the reaction with PNA as immobilized agent shows that the reducing end is cleaved only if Endo-βGalactosidase is used (reactions 1 and 3). This indicates that the Endo-βGalactosidase site is located between the site for PNA and the reducing end. On the other hand, the Fucosidase site must be located between the PNA site and the other end of the saccharide.

When taking into account the above data, it is now possible to propose a sequence of the saccharide as follows:

Fucα(1-3,1-4)GlcNAc(1-3)Gal(1-4)Glc/GlcNAc—reducing end

The above experiment clearly demonstrates that the method of the invention can yield a variety of data, including sequence information, based upon relatively few reactions. Some details in the sequence information may not be complete, such as the (1-3) or (14) connection between Fucose and GlcNAc in the above saccharide. Had the monosaccharide composition of the pentasaccharide been known, then the above analysis would have yielded all of the details of the pentasaccharide. Nevertheless, the information gained even in the absence of the monosaccharide composition data is very precise compared to prior art methods.

### Example 4

### Derivation of Partial or Complete Sequence Information

The method of the invention is suitable for automation. Thus, the steps described above, for example, in examples 1 to 3, may be carried out using an automated system for mixing, aliquoting, reacting, and detection. The data obtained by such an automated process may then be further processed in order to “collapse” the mapping information to partial or complete sequence information. The method for such data processing is described in further detail below.

After all data have been collected, a comparison is made between detection signals obtained from reactions prior to the addition of glycosidase, to signals obtained after the addition (and reaction with) of glycosidase. Those signals that disappear after reaction with glycosidase are marked. This may advantageously be done by preparing a list of those signals, referred to hereinafter as a first list. The identity of two sites on the polysaccharide may now be established for each such data entry. The position in the (optionally virtual) array indicates the first essentially sequence-specific agent. If a signal has been detected before reaction with the glycosidase, the recognition site for that agent must exist in the polysaccharide. The disappearance of a signal, for instance, of the signal associated with the second essentially sequence-specific agent, now indicates that the glycosidase cleaves between the recognition sites of the first and second essentially sequence-specific agents. The sequence of recognition sites is therefore (first essentially sequence-specific agent)-(glycosidase) second essentially sequence-specific agent). If the signal for the reducing end is still present after digestion with the glycosidase, then the relative order of the recognition sequences with respect to the reducing end can be established; otherwise, both possibilities (a-b-c and c-b-a) must be taken into account. For the purpose of illustration, the term “recognition site of the first essentially sequence-specific agent” shall be denoted in the following “first recognition site”, the term “recognition site for the second essentially sequence-specific agent” shall be denoted “second recognition site”, and the term “recognition site for glycosidase” shall be denoted “glycosidase”.

It is now possible to create a second list of triplets of recognition sites of the above type (type 1 triplets):

(first recognition site)-(glycosidase)-(second recognition site).

Similarly, a third list can now be created relating to (optionally virtual) array locations where all signals remain after addition of glycosidase (type 2 triplets):

(glycosidase)-(first recognition site)-(second recognition site)

Obviously, a sufficient number of triplets defines a molecule in terms of its sequence, i.e., there can only be one sequence of saccharides that will contain all of the triplets found. A lower number of triplets may be required when information on the length of the molecule is available. The number of required triplets may be even lower if the total sugar content of the molecule is known. Both saccharide molecular weight and total monosaccharide content may be derived from prior art methods well known to the skilled person.

The process of obtaining sequence information, i.e., of collapsing the triplets into a map of recognition sites, is described below.

The second and third lists of triplet recognition sites are evaluated for identity (three out of three recognition sites identical), high similarity (two out of three recognition sites identical), and low similarity (one out of three recognition sites identical). For the purposes of illustration, it is now assumed that the polysaccharide is a linear polysaccharide, such as, for example, the saccharide portion of the glycan heparin.

The above second and third lists are then used to prepare therefrom a set of lists of triplets wherein each list in the set of lists contains triplets that share the same glycosidase recognition sequence. By comparing all triplets containing a certain glycosidase recognition sequence with all triplets containing a second glycosidase recognition sequence, it is now possible to divide the polysaccharide sequence into four areas, ranging from the first end of the molecule to glycosidase 1 (fragment a), from glycosidase 1 to glycosidase 2 (fragment b), and from glycosidase 2 to the second end of the molecule (fragment c):

<first end><glycosidase 1><glycosidase2><second end>

Identical recognition sites within triplets of type 2 with different glycosidase sites, wherein the recognition sites are located in the same direction in relation to the respective glycosidase site, are candidates for the location within either the area a or c, depending on the location. Identical recognition sites within triplets of type 2 with different glycosidase sites, wherein the recognition sites are located in different directions (e.g. one in the direction of the reducing end, in the other triplet, in the direction of the non-reducing end), are candidates for the location within the area b, i.e., between the two glycosidase sites.

Identical recognition sites within triplets of type 1 with different glycosidase sites are candidates for the location of one of the first or second recognition sites in area a (or c), and the other of the first or second recognition sites being located in the area c (or a). That is, if one of the first or second recognition sites is located in area a, then the other of the first or second recognition sites must be located in area b, and vice versa. None of the the first or second recognition sites may be located in area b.

Identical recognition sites within triplets of type 1 with different glycosidase sites, wherein a given recognition site is located in one of the triplets, in the direction of the reducing end and in the other triplet, in the direction of the non-reducing, are candidates for the location of the recognition site within area b.

Having established the above positional relationships for a number of recognition sites within the triplets, the total of the recognition sequences can now be arranged in a certain order using logical reasoning. This stage is referred to as a sequence map. If a sufficient number of recognition sequences are arranged, the full sequence of the saccharide may be derived therefrom. As the method does not determine the molecular weight of the saccharide, the chain length is unknown. Therefore, if the degree of overlap between the various recognition sites is insufficient, there may be regions in the sequence where additional saccharide units may be present. Such saccharide units may be undetected if they do not fall within a recognition site of any of the essentially sequence-specific agents used. However, the entire sequence information may also be obtained in this case, by first obtaining the molecular weight of the saccharide, which indicates its chain length, and secondly its total monosaccharide content.

Another possibility of closing gaps in the sequence map is the method of example 2, wherein sequential degradation by glycosidase is employed to derive sequence information.

The existence of branching points in the saccharide may complicate the method as outline above. One remedy to that is to use glycosidases to prepare fractions of the molecule, and analyze these partial structures. The extent of branching in such partial structures is obviously lower than in the entire molecule. In addition, reagents may be employed that specifically recognize branching points. Examples for such reagents are e.g., the antibodies employed in example 1 above. Each of these antibodies binds a saccharide sequence that contains at least one branching point. Moreover, certain enzymes and lectins are available that recognize branched saccharide structures. For instance, the enzyme pullanase (EC 3.2.1.41) recognizes a branched structure. In addition, antibodies may be generated by using branched saccharide structures as antigens. Moreover, it is possible to generate peptides that bind certain saccharide structures, including branched structures (see e.g., Deng S J, MacKenzie C R, Sadowska J, Michniewicz J, Young N M, Bundle D R, Narang; Selection of antibody single-chain variable fragments with improved carbohydrate binding by phage display. J. Biol. Chem. 269, 9533-38, 1994).

In addition, knowledge of the structure of existing carbohydrates will in many cases predict accurately the existence of branching points. For instance, N-linked glycans possess a limited number of structures, as listed at p. 6 of the oxford Glycosystems catalog. These structures range from monoantennary to pentaantennary. The more complicated structures resemble simpler structures with additional saccharide residues added. Therefore, if monoantennary structure is identified, it is possible to predict all of the branching points in a more complicated structure, simply by identifying the additional residues and comparing these data with a library of N-linked glycan structures.

Moreover, it will often be possible by analyzing data gathered according to the method of the invention, to deduce the existence and location of branching points logically. For instance, if two recognition sites, denoted a and b, are located on different branches, then digesting with a glycosidase whose site is located between the reducing end and the branching point will result in loss of the reducing end marker. The markers for both recognition sites a and b, however, will remain. If a glycosidase located between the branching point and recognition site a is used, then the marker for recognition site b and the reducing end marker will be cleaved off. Not taking into account the possibility of branching points, this would indicate that the recognition site b is located between the recognition site a and the reducing end. However, if a glycosidase located between the recognition site b and the branching point is used, the reducing end marker and recognition site a will be cleaved off. Again, not taking into account the possibility of branching, this would indicate that recognition site a is located between the reducing end and recognition site b. These deductions are obviously incompatible with one another, and can only be resolved if one assumes that recognition sites a and b are located on two different branches. The branching point is located between the recognition sites a and b and the first of the above glycosidases. The other above glycosidases used are located on a branch each, between the branching point and the respective recognition site (a or b).

Therefore, when using agents that recognize branched structures in the method of the invention, as essentially sequence-specific agents, it is possible to derive information on the existence and location of branching points in the saccharide molecule. This information can then be used to construct sequence maps of each branch of the structure, yielding a sequence map of the entire branched structure. The gaps in such a structure may then be closed as in the case of unbranched saccharides, according to the invention, i.e., by using additional reactions, by digestion with glycosidases, whereby the regions of the molecule where gaps exist are specifically isolated for further analysis according to the method of the invention, and by sequential glycosidase digestion as described further above.

In summary, a method for determining the sequence of a saccharide and/or for mapping the structure of the saccharide according to the invention comprises the steps of:

1. collecting triplets of type 1 and type 2

2. sorting the triplets according to similarity

3. comparing triplets with different glycosidase recognition sites

4. arranging the triplets in the order of occurrence on the saccharide

5. arranging the glycosidase recognition sites

6. checking the compatibility to the triplets

7. arranging recognition sequences of glycosidases and of first and second essentially sequence-specific agents in a single file order

8. translating the recognition sequences (sites) into polysaccharide sequence

9. correcting “overlap” problems

10. outputting a sequence

11. checking against all available data

After the above step 5 has been carried out, a preliminary order of glycosidase sites has been established. In step 6, it is now checked for each triplet whether predictions based thereon are in agreement with that order. Then, based on contradiction in the data, a new model is generated that fits the data of the triplet. This model is then tested against the data of all triplets. Furthermore, additional reactions may be carried out, in order to extract additional vectorial information regarding the recognition sites that involve the triplet.

After the above step 8, wherein the sequentially arranged recognition sites are translated into a sequence of actual monosaccharide units, a model of the saccharide sequence can be suggested. In order to test the model, a number of questions needs to be answered. The first of these is, what is the minimum sequence that would still have the same sequence map? At this stage, information on molecular weight and monosaccharide composition, if available, is not taken into account. This approach merely serves the creation of a sequence which incorporates all of the available data with as few as possible contradictions. In that respect, the second question to be answered is, does the minimum sequence still agree with all of the data available at that point (excluding optional molecular weight and monosaccharide composition data)? The third question to be answered is, do other sequences exist that would fit the sequence map as established? In the affirmative, the additional sequences may then be tested using the question: How does each sequence model agree with the triplet information, and with additional optional data, such as information on the molecular weight, monosaccharide composition, and model saccharide structures known from biology.

Finally, the sequence model that has been found to be best according to the steps 1-10 described above, will then be tested against all triplets, monosaccharide composition, prior knowledge on the molecular weight and structural composition of the saccharide, and predictions from biologically existent similar structures. By such repeated testing, the contradictions between the available data and the sequence model are identified, and if possible, the sequence model is adapted to better represent the data.

### EXAMPLE 5

### Glycomolecule Identity (GMID) Analysis of Milk Samples

The aim of this example is to demonstrate the application of the GMID technique to the analysis and comparison of milk samples.

A. Membranes and 1^{st} Layer Lectins:

The supporting surface used in the experiments described hereinbelow is a nitrocellulose membrane. The membranes were prepared as follows:

1. Nitrocellulose membranes were cut out and their top surface marked out into an array of 9×6 squares (3 mm² each square). The membranes were then placed on absorbent paper and the top left square of each one marked with a pen.

2. Lyophilized lectins were resuspended in water to a final concentration of 1 mg/ml. The resuspended lectins (and a control solution; 5% bovine serum albumin) were vortex mixed and 1 μl of each solution is added to one of the 28 squares on the blot, indicated by shading in the following illustrative representation of a typical blot:

[image]

TABLE 11

| Lectin | Manufacturer | Cat. No. |
|---|---|---|
| WGA | Vector | MK2000 |
| SBA | Vector | MK2000 |
| PNA | Vector | MK2000 |
| DBA | Vector | MK2000 |
| UEA I | Vector | MK2000 |
| CON A | Vector | MK2000 |
| RCA I | Vector | MK2000 |
| BSL I | Vector | MK3000 |
| SJA | Vector | MK3000 |
| LCA | Vector | MK3000 |
| Swga | Vector | MK3000 |
| PHA-L | Vector | MK3000 |
| PSA | Vector | MK3000 |
| AAA | — | — |
| PHA-E | Vector | MK3000 |
| PNA | Leuven | LE-408 |
| LCA | Sigma | L9267 |
| DSA | Sigma | L2766 |
| APA | | — |
| WGA | Leuven | LE-429 |
| Jacalin | Leuven | LE-435 |
| 5% BSA | Savyon | M121-033 |

3. The prepared blots were placed in 90 mm petri dishes.

4. The blots were blocked by adding to each petri dish 10 ml of any suitable blocking solution well known to the skilled artisan (e.g. 5% bovine serine albumin).

5. The dishes containing the blots in the blocking solution were agitated gently by rotation on a rotating table (50 rpm) for 2 hours at room temperature (or overnight at 4° C., without rotation).

6. The blots were then washed by addition of 10 ml washing solution to each petri dish. Any commonly available buffered solution (e.g. phosphate buffered saline) may be used for performing the washing steps. The dishes were washed by rotating gently (50 rpm) for 5 minutes. The procedure was performed a total of three times, discarding the old washing solution and replacing with fresh solution each time.

B: Addition of Milk Samples:

The milk samples used were as follows:

1. Bovine UHT long-life milk (3% fat) obtained from Ramat haGolan dairies, Israel (lot 522104);

2. Pasteurized goat's milk, obtained from Mechek dairies, Israel (lots 1 and 2);

3. Non-pasteurized goat's milked obtained as in 2. (lots 3 and 4).

The milk samples were diluted to 10% v/v and approximately 5 ml of each sample applied to separate blots.

Duplicate blots were prepared for each of the aforementioned milk samples. In addition a further pair of blots were prepared without the addition of saccharides (negative control).

The blots were then incubated at room temperature with agitation for one hour. C. Colored lectins:

From prior knowledge of the monosaccharide composition of the milks tested, and by application of a computer program based on the algorithm described hereinbelow in Example 7, the following colored lectins were chosen: Con A, WA.

A mixture of these two lectins was prepared in washing solution, such that the concentration of each colored lectin was 2 mg/ml.

500 μl of each lectin mix was incubated on the blots prepared as described above. Each blot was read both by measuring the fluorescence of fluorescein at 520 nm, and, in the case of the biotinylated lectin, measuring the signal of the TMB blue color produced following reaction of biotin with an HRP-streptavidin solution

The results obtained for the FITC-labeled and biotin-labeled lectins are given in Tables 12 and 13, respectively. The results presented in these tables are measured on a 0 to 3 scale, wherein 0 represents a signal that is below the noise level, and wherein results of 1-3 represent positive signals (above noise) following subtraction of the results obtained in the no-saccharide control.

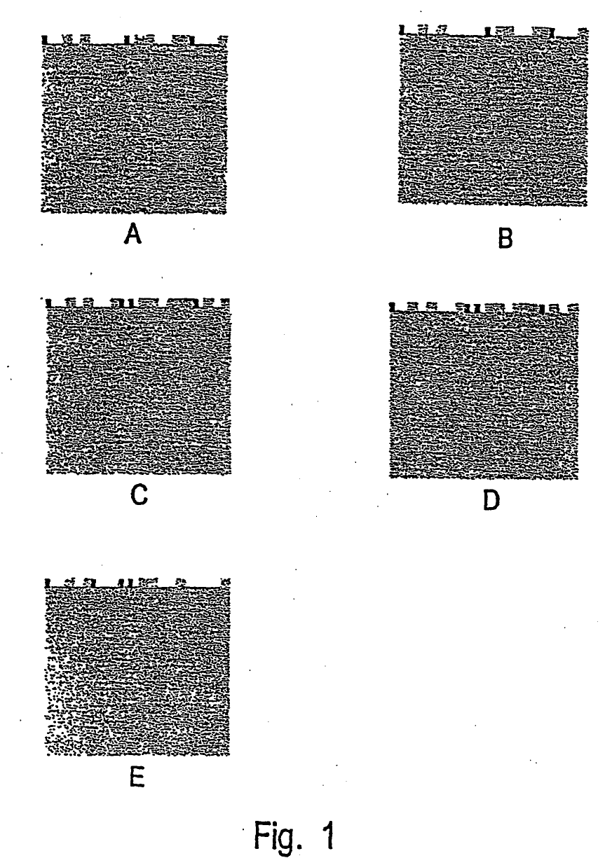

Glycomolecule identity (GMID) cards obtained from these results for pasteurized goat's milk (lots 1 and 2), non-pasteurized goat's milk (lots 3 and 4) and bovine milk are shown in FIG. 1 (A to E, respectively). The positions of lectins 1 to 24 are shown in one row from left to right at the top of each card 1.

D. Interpretation of Results:

The bovine milk sample yielded a GMID indicating that the polysaccharide in the sample contains saccharides that yield positive results for lectins specific for:

a. glucose/mannose (ConA, PSA and LCA);

b. GlcNac (WGA and DSA).

The pasteurized goat milk samples yielded positive results for:

a. glucose/mannose (conA, PSA and LCA);

b. GlcNac (DSA).

No difference in lectin reactivity between the lots tested was observed. The non-pasteurized goat milk sample gave a positive reaction for:

a. glucose/mannose (ConA, PSA and LCA);

b. GlcNac (DSA).

In summary, the bovine milk differed from the goat's milk in that only the former reacted with WGA. There was essentially no difference between the pasteurized and non-pasteurized goat's milk samples, with the exception that the signal intensity was significantly lower in the pasteurized samples.

### EXAMPLE 6

Glycomolecule Identity (GMID) Analysis of Lipopolysaccharides

A GMID analysis was performed on five different bacterial lipopolysaccharides obtained from Sigma Chemical Co. (St. Louis, Mo., USA)(LPS#1, 7, 10, 15 and 16), essentially using the method as described in Example 5, above. The colored lectins used were ECL, WGA, WA and SBA.

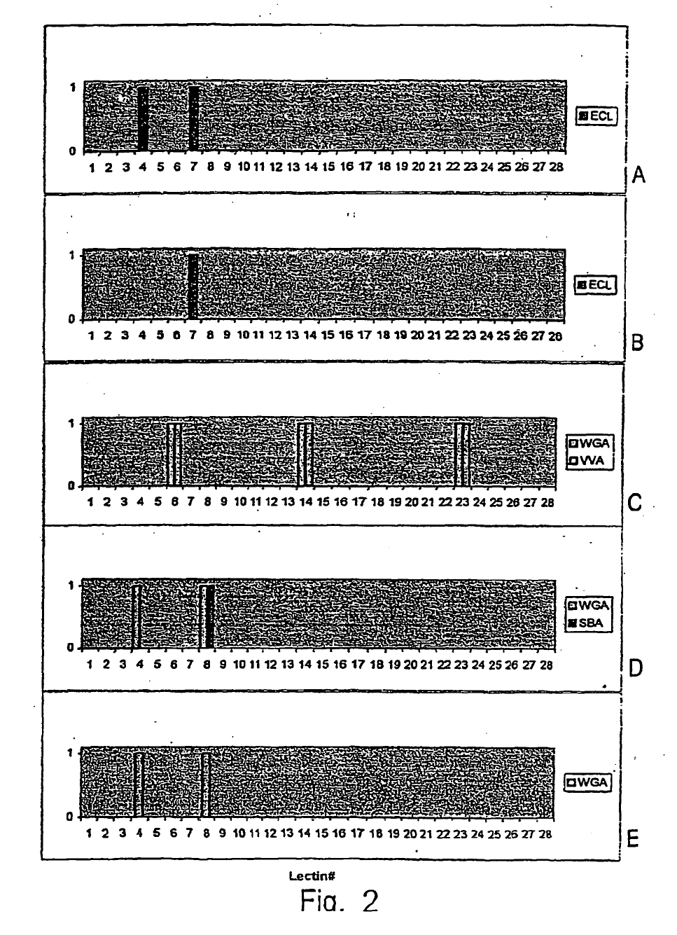

The GM”) cards obtained for samples LPS# 1, 7, 10, 15 and 16 are shown in FIG. 2 (A to E, respectively). It may be seen from this figure that the GMID cards provide unique “fingerprints” for each of the different lipopolysaccharides, and may be used for identifying the presence of these compounds in samples containing bacteria or mixtures of their products.

### EXAMPLE 7

Method for Selecting Colored Lectins

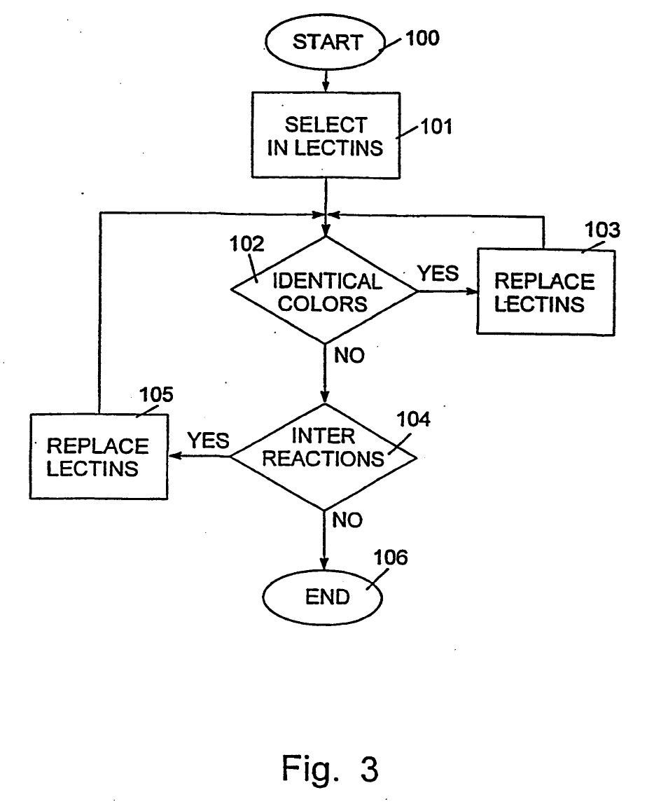

A number of factors must be taken into consideration when selecting colored lectins for use in the method of polysaccharide analysis illustrated in Examples 5 and 6. Among these considerations are the need for each of the chosen lectins to have a distinguishable color or other detectable marker, and for the need to reduce interactions between lectins. A flow chart illustrating an algorithm for use in colored marker selection is shown in FIG. 3. The algorithm shown in FIG. 3 begins with the selection of n colored lectins (or other detectable markers) 101, the initial selection being made in accordance with information obtained about the partial or full monosaccharide composition of the saccharide to be analyzed.

In the next step 102, the colors of the selected lectins are examined in order to check for identity/non-identity of the colors selected. If there are identical colors in the selected group, then the process proceeds to step 103, otherwise the flow proceeds with step 104. In step 103, one of the lectins that has been found to have a non-unique color is replaced by another lectin that belongs to the same binding category (that is, one that has the same monosaccharide binding specificity); the flow proceeds to step 102.

In step 104, the n selected lectins are tested in order to detect any cross-reactivity with each other, and with the non-colored lectins used in the first stage of the method described hereinabove in Example 5. If cross-reactivity is found, then the process continues to step 105, otherwise the flow proceeds to step 106, where the algorithm ends.

In step 105, one of the lectins determined to cross-react with another lectin is replaced by a lectin which does not cross-react; the flow then proceeds to 102. The algorithm ends with step 106.

It is to be emphasized that while for values of n which are small, and for saccharides with a simple monosaccharide composition, the above-described algorithm may be applied by the operator himself/herself manually working through each step of the selection procedure. Alternatively (and especially for cases where n is a larger number or the monosaccharide composition is more complex), the algorithmic processes described hereinabove may be performed by a computer program designed to execute the processes.

The above examples have demonstrated the usefulness of the method described herein. However, they have been added for the purpose of illustration only. It is clear to the skilled person that many variations in the essentially sequence-specific agents used, in the reaction conditions therefor, in the technique of immobilization, and in the sequence of labeling, reaction and detection steps may be effected, all without exceeding the scope of the invention.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the relatedness of a first glycoprotein and a second glycoprotein, the method comprising:
providing a first fingerprint of a first glycoprotein, wherein the first fingerprint comprises binding information for at least a first saccharide-binding agent and a second saccharide-binding agent for the first glycoprotein;
providing a second fingerprint of a second glycoprotein, wherein the second fingerprint comprises binding information for at least the first saccharide-binding agent and the second saccharide-binding agent for the second glycoprotein;
comparing the first fingerprint and the second fingerprint, wherein said comparing comprises determining whether the first glycoprotein and the second glycoprotein bind to the first saccharide binding agent, and whether the first glycoprotein and the second glycoprotein bind to the second saccharide binding agent,
thereby determining the relatedness of the first glycoprotein and second glycoprotein.

2. The method of claim 1, wherein the first fingerprint is identified by a method comprising
providing a first glycoprotein comprising a first carbohydrate polymer,
contacting the first carbohydrate polymer with the first saccharide-binding agent;
determining whether the first carbohydrate polymer binds to the first saccharide-binding agent;
contacting said carbohydrate polymer with the second saccharide-binding agent, wherein the second saccharide-binding agent comprises a detectable label; and
determining whether the first carbohydrate polymer binds to the second saccharide-binding reagent,
thereby generating a fingerprint of the first glycoprotein.

3. The method of claim 2, wherein the second fingerprint is identified by a method comprising
providing a second glycoprotein comprising a second carbohydrate polymer,
contacting said carbohydrate polymer with the first saccharide-binding agent;
determining whether the second carbohydrate polymer binds to said saccharide-binding agent;
contacting the second carbohydrate polymer with the second saccharide-binding agent, wherein the second saccharide-binding agent comprises a detectable label; and
determining whether said carbohydrate polymer binds to the second saccharide-binding reagent,
thereby generating a fingerprint of the second glycoprotein.

4. The method of claim 2, further comprising contacting the first carbohydrate polymer with at least five saccharide-binding agents, and determining whether said carbohydrate polymer binds to each of said at least five saccharide-binding reagents.

5. The method of claim 2, wherein binding of the first and second saccharide-agent is determined by
a) providing a surface comprising at least one first saccharide-binding agent attached to a predetermined location on said surface;
b) contacting said surface with a carbohydrate polymer under conditions allowing for the formation of a first complex between the first saccharide-binding agent and said carbohydrate polymer;
c) contacting said surface with at least one second saccharide-binding agent under conditions allowing for formation of a second complex between the first complex and the second saccharide-binding agent; and
d) identifying the first saccharide-binding agent and second saccharide-binding agent in the second complex.

6. The method of claim 2, wherein the second saccharide-binding agent further comprises a detectable label and the second saccharide binding agent is identified by detecting said label and the first saccharide binding agent is identified by determining the location of the detected label on the substrate.

7. The method of claim 6, wherein said detectable label is selected from the group consisting of a chromogenic label, a radiolabel, a fluorescent label, and a biotinylated label.

8. The method of claim 6, wherein said surface comprises at least five saccharide-binding agents affixed to said surface.

9. The method of claim 6, wherein said surface is contacted with at least 5 second saccharide-binding agents.

10. The method of claim 6, wherein said surface is contacted with at least five second saccharide-binding agents.

11. The method of claim 6, wherein the first saccharide binding agent is selected from the group consisting of a lectin, a saccharide-cleaving enzyme, and an antibody to a saccharide.

12. The method of claim 6, wherein the second saccharide binding agent is selected from the group consisting of a lectin, a polysaccharide-cleaving or modifying enzyme, and an antibody to a saccharide.

13. The method of claim 6, wherein said carbohydrate polymer is provided after digestion with a saccharide-cleaving agent.

14. The method of claim 6, wherein said carbohydrate polymer is digested with a saccharide-cleaving agent prior to contacting said saccharide with the second saccharide-binding agent.

15. The method of claim 1, wherein the first fingerprint and second fingerprint comprise information for at least five saccharide-binding agents.

16. The method of claim 1, wherein the test glycoprotein is present in a biological fluid.

17. The method of claim 16, wherein the biological fluid is selected from the group consisting of blood, serum, urine, saliva, milk, ductal fluid, tears and semen.

18. A method for identifying a glycoprotein, the method comprising:
providing a first fingerprint of a test glycoprotein, wherein the first fingerprint comprises binding information for at least a first saccharide-binding agent and a second saccharide-binding agent for the first glycoprotein;
comparing the first fingerprint to at least one reference fingerprint, wherein the reference glycoprotein fingerprint comprises binding information for at least the first saccharide-binding agent and the second saccharide-binding agent for at least one reference glycoprotein,
thereby identifying the test glycoprotein.

19. The method of claim 18, wherein the first fingerprint and reference fingerprint comprise information for at least five saccharide-binding agents.

20. The method of claim 18, wherein the first fingerprint is identified by a method comprising
providing a first glycoprotein comprising a first carbohydrate polymer,
contacting the first carbohydrate polymer with the first saccharide-binding agent;
determining whether the first carbohydrate polymer binds to the first saccharide-binding agent;
contacting said carbohydrate polymer with the second saccharide-binding agent, wherein the second saccharide-binding agent comprises a detectable label; and
determining whether the first carbohydrate polymer binds to the second saccharide-binding reagent,
thereby generating a fingerprint of the first glycoprotein.

21. The method of claim 18, wherein the reference fingerprint is identified by a method comprising
providing a second glycoprotein comprising a second carbohydrate polymer,
contacting said carbohydrate polymer with the first saccharide-binding agent;
determining whether the second carbohydrate polymer binds to said saccharide-binding agent;
contacting the second carbohydrate polymer with the second saccharide-binding agent, wherein the second saccharide-binding agent comprises a detectable label; and
determining whether said carbohydrate polymer binds to the second saccharide-binding reagent,
thereby generating a fingerprint of the second glycoprotein.

22. The method of claim 21, further comprising contacting the first carbohydrate polymer with at least five saccharide-binding agents, and determining whether said carbohydrate polymer binds to each of said at least five saccharide-binding reagents.

23. The method of claim 22, wherein binding of the first and second saccharide-agent is determined by
a) providing a surface comprising at least one first saccharide-binding agent attached to a predetermined location on said surface;
b) contacting said surface with a carbohydrate polymer under conditions allowing for the formation of a first complex between the first saccharide-binding agent and said carbohydrate polymer;
c) contacting said surface with at least one second saccharide-binding agent under conditions allowing for formation of a second complex between the first complex and the second saccharide-binding agent; and
d) identifying the first saccharide-binding agent and second saccharide-binding agent in the second complex.

24. The method of claim 23, wherein the second saccharide-binding agent further comprises a detectable label and the second saccharide binding agent is identified by detecting said label and the first saccharide binding agent is identified by determining the location of the detected label on the substrate.

25. The method of claim 24, wherein said detectable label is selected from the group consisting of a chromogenic label, a radiolabel, a fluorescent label, and a biotinylated label.

26. The method of claim 23, wherein said surface comprises at least five saccharide-binding agents affixed to said surface.

27. The method of claim 23, wherein said surface is contacted with at least 5 second saccharide-binding agents.

28. The method of claim 26, wherein said surface is contacted with at least five second saccharide-binding agents.

29. The method of claim 23, wherein the first saccharide binding agent is selected from the group consisting of a lectin, a saccharide-cleaving enzyme, and an antibody to a saccharide.

30. The method of claim 23, wherein the second saccharide binding agent is selected from the group consisting of a lectin, a polysaccharide-cleaving or modifying enzyme, and an antibody to a saccharide.

31. The method of claim 23, wherein said carbohydrate polymer is provided after digestion with a saccharide-cleaving agent.

32. The method of claim 25, wherein said carbohydrate polymer is digested with a saccharide-cleaving agent prior to contacting said saccharide with the second saccharide-binding agent.

33. A method of modifying a glycoprotein, the method comprising:
providing a first fingerprint of a test glycoprotein, wherein the first fingerprint comprises binding information for at least a first saccharide-binding agent and a second saccharide-binding agent for the first glycoprotein;
comparing the first fingerprint to at least one reference fingerprint, wherein the reference fingerprint comprises binding information for at least the first saccharide-binding agent and the second saccharide-binding agent for at least one reference glycoprotein;
identifying differences in the first fingerprint and the reference fingerprint; and
altering the test glycoprotein to decrease or increase the differences in the first fingerprint and reference fingerprint, thereby modifying said glycoprotein.

34. The method of claim 33, wherein said altering decreases the difference between the first fingerprint and reference fingerprint.

35. The method of claim 33, wherein said altering increases the difference between the first fingerprint and reference fingerprint.

36. The method of claim 33, wherein said altering comprises generating a fingerprint of said altered test glycoprotein and comparing the fingerprint of said altered test glycoprotein to the reference fingerprint.

37. A method for determining the relatedness of a first polysaccharide and a second polysaccharide, the method comprising:
providing a first fingerprint of a first polysaccharide, wherein the first fingerprint comprises binding information for at least a first saccharide-binding agent and a second saccharide-binding agent for the first polysaccharide;
providing a second fingerprint of a second polysaccharide, wherein the second fingerprint comprises binding information for at least the first saccharide-binding agent and the second saccharide-binding agent for the second polysaccharide;
comparing the first fingerprint and the second fingerprint, wherein said comparing comprises determining whether the first polysaccharide and the second polysaccharide bind to the first saccharide binding agent, and whether the first polysaccharide and the second polysaccharide bind to the second saccharide binding agent, thereby determining the relatedness of the first and second polysaccharide.

38. The method of claim 37, wherein said first polysaccharide is provided as a fragment of a larger polysaccharide.

39. The method of claim 37, wherein the second polysaccharide is associated with a known biological property.

40. The method of claim 37, wherein the first fingerprint and second fingerprint comprise information for at least five saccharide-binding agents.

41. The method of claim 37, wherein the first fingerprint is identified by a method comprising
providing said first polysaccharide,
contacting the first polysaccharide with the first saccharide-binding agent;
determining whether the first polysaccharide binds to the first saccharide-binding agent;
contacting said first polysaccharide with the second saccharide-binding agent, wherein the second saccharide-binding agent comprises a detectable label; and
determining whether the first polysaccharide binds to the second saccharide-binding reagent,
thereby generating a fingerprint of the first polysaccharide.

42. The method of claim 41, wherein the second fingerprint is identified by a method comprising
providing a second polysaccharide comprising a second carbohydrate polymer,
contacting said carbohydrate polymer with the first saccharide-binding agent;
determining whether the second carbohydrate polymer binds to said saccharide-binding agent;
contacting the second carbohydrate polymer with the second saccharide-binding agent, wherein the second saccharide-binding agent comprises a detectable label; and
determining whether said carbohydrate polymer binds to the second saccharide-binding reagent,
thereby generating a fingerprint of the second polysaccharide.

43. The method of claim 41, further comprising contacting the first carbohydrate polymer with at least five saccharide-binding agents, and determining whether said carbohydrate polymer binds to each of said at least five saccharide-binding reagents.

44. The method of claim 41, wherein binding of the first and second saccharide-agent is determined by
a) providing a surface comprising at least one first saccharide-binding agent attached to a predetermined location on said surface;
b) contacting said surface with a carbohydrate polymer under conditions allowing for the formation of a first complex between the first saccharide-binding agent and said carbohydrate polymer;
c) contacting said surface with at least one second saccharide-binding agent under conditions allowing for formation of a second complex between the first complex and the second saccharide-binding agent; and
d) identifying the first saccharide-binding agent and second saccharide-binding agent in the second complex.

45. The method of claim 44, wherein the second saccharide-binding agent further comprises a detectable label and the second saccharide binding agent is identified by detecting said label and the first saccharide binding agent is identified by determining the location of the detected label on the substrate.

46. The method of claim 45, wherein said detectable label is selected from the group consisting of a chromogenic label, a radiolabel, a fluorescent label, and a biotinylated label.

47. The method of claim 44, wherein said surface comprises at least five saccharide-binding agents affixed to said surface.

48. The method of claim 44, wherein said surface is contacted with at least 5 second saccharide-binding agents.

49. The method of claim 44, wherein said surface is contacted with at least five second saccharide-binding agents.

50. The method of claim 44, wherein the first saccharide binding agent is selected from the group consisting of a lectin, a saccharide-cleaving enzyme, and an antibody to a saccharide.

51 The method of claim 44, wherein the second saccharide binding agent is selected from the group consisting of a lectin, a polysaccharide-cleaving or modifying enzyme, and an antibody to a saccharide.

52. The method of claim 44, wherein the first polysaccharide is provided after digestion with a saccharide-cleaving agent.

53. The method of claim 44, wherein the first polysaccharide is digested with a saccharide-cleaving agent prior to contacting said first polysaccharide with the second saccharide-binding agent.

54. A method for modifying a polysaccharide, the method comprising:
providing a first fingerprint of a test polysaccharide, wherein the first fingerprint comprises binding information for at least a first saccharide-binding agent and a second saccharide-binding agent for the first polysaccharide;
comparing the first fingerprint to at least one reference fingerprint, wherein the reference fingerprint comprises binding information for at least the first saccharide-binding agent and the second saccharide-binding agent for at least one reference polysaccharide;
identifying differences in the first fingerprint and the reference fingerprint; and
altering the test polysaccharide to decrease or increase the differences in the first fingerprint and reference fingerprint, thereby modifying said polysaccharide.

55. The method of claim 54, wherein said altering comprises generating a fingerprint of said altered test polysaccharide and comparing the fingerprint of said altered test polysaccharide to the reference fingerprint.

56. A polysaccharide produced by the method of 54.

57. A plurality of polysaccharides comprising the polysaccharide of claim 56.

58. A substrate comprising the plurality of claim 57.

59. A method of diagnosing a pathology associated with a carbohydrate polymer in a subject, the method comprising:
providing a test fingerprint of a carbohydrate polymer from a subject suspected of having said pathology; and
comparing the test fingerprint with a reference fingerprint, wherein the test fingerprint is from a carbohydrate polymer in a reference sample whose pathology state is known, wherein a correspondence between the test fingerprint and the reference fingerprint indicates the subject and the reference sample have the same pathology state.

60. The method of claim 59, wherein the reference sample comprises a database.

61. The method of claim 59, wherein said carbohydrate polymer is a glycoprotein, a polysaccharide, or a glycolipid.

62. The method of claim 59, wherein the subject is a human.

63. A method of identifying a function associated with a carbohydrate polymer, the method comprising:
providing a test fingerprint of a carbohydrate polymer from a test sample; and
comparing the test fingerprint with a reference fingerprint, wherein the test fingerprint is from a carbohydrate polymer whose functional status is known,
wherein a correspondence between the test fingerprint and the reference fingerprint indicates the subject and the reference sample have the same functional status.

64. The method of claim 63, wherein the carbohydrate polymer is a glycoprotein, polysaccharide, or a glycolipid.

65. The method of claim 63, wherein said carbohydrate polymer is a glycoprotein.

66. A method of identifying a carbohydrate polymer, the method comprising
providing a test fingerprint of a carbohydrate polymer; and
comparing the test fingerprint with a reference fingerprint, wherein the test fingerprint is from a reference carbohydrate polymer whose identity is known,
wherein a correspondence between the test fingerprint and the reference fingerprint indicates the subject and the reference carbohydrate sample are the same.

67. A method of identifying an agent that modulates the structure of a carbohydrate polymer, the method comprising;
providing a biological sample comprising said carbohydrate polymer;
contacting the sample with a test agent;
identifying a carbohydrate polymer fingerprint of one or more carbohydrate polymers in said sample;
comparing the carbohydrate polymer fingerprint to a carbohydrate polymer fingerprint of said one or more carbohydrate polymers in a sample that is not contacted with said agent;
identifying a difference in the carbohydrate fingerprint profiles, if present, in the test and reference fingerprints,
thereby identifying an agent that modulates the structure of a carbohydrate polymer.

68. A method of identifying a candidate therapeutic agent for a pathophysiology associated with a carbohydrate polymer, the method comprising
providing a test biological sample comprising a cell capable of expressing said carbohydrate polymer;
contacting the test biological sample with a test agent;
identifying a carbohydrate polymer fingerprint of one or more carbohydrate polymers in said biological sample;
comparing the carbohydrate polymer fingerprint to a carbohydrate polymer fingerprint of one or more carbohydrate polymers in a reference biological sample comprising at least one cell whose pathophysiological status is known; and
identifying a difference in the carbohydrate finger profiles, if present, in the test biological sample and reference biological sample,
thereby identifying a therapeutic agent for a pathophysiology associated with the carbohydrate polymer.

69. A method of identifying an individualized therapeutic agent suitable for treating a pathophysiology associated with a carbohydrate polymer in a subject, the method comprising:
providing from said subject a biological sample comprising said carbohydrate polymer;
contacting the test biological sample with a test agent;
identifying a carbohydrate polymer fingerprint of one or more carbohydrate polymers in said biological sample;
comparing the carbohydrate polymer fingerprint to a carbohydrate polymer fingerprint of said one or more carbohydrate polymers in a reference biological sample whose pathophysiological status is known; and
identifying a difference in the carbohydrate finger profiles, if present, in the test biological sample and reference biological sample,
thereby identifying an individualized therapeutic agent for said subject.

70. A method of assessing the efficacy of a treatment of pathophysiology associated with a carbohydrate polymer, the method comprising:
providing from the subject a test biological sample comprising said carbohydrate polymer;
determining a carbohydrate fingerprint of said carbohydrate polymer; and
comparing the carbohydrate fingerprint of said polymer with a reference carbohydrate polymer fingerprint, wherein the reference carbohydrate polymer fingerprint is derived from a carbohydrate polymer whose pathophysiological status is known;
thereby assessing the efficacy of treatment of the pathophysiology in the subject.

71. A method of treating a pathophysiology associated with a carbohydrate polymer mediated pathway in a subject, the method comprising administering to the subject an agent that modulates a carbohydrate polymer in said patient, wherein said modulation alters a carbohydrate polymer fingerprint in said patient.
